(19) United States
(12) Patent Application Publication
(10) Pub. No.: US 2010/0047819 A1
(43) Pub. Date: Feb. 25, 2010

(54) MULTIPLEX ASSAY FOR RHEUMATOID ARTHRITIS

(75) Inventors: Heidi Scholz, Vacaville, CA (US); Greg Marr, Martinez, CA (US); Craig Lingenfelter, Pleasant Hill, CA (US); Sandra Garcia, Concord, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BIO-RAD LABORATORIES, INC., Hercules, CA (US)

(21) Appl. No.: 12/542,434

(22) Filed: Aug. 17, 2009

Related U.S. Application Data
(60) Provisional application No. 61/090,082, filed on Aug. 19, 2008.

(51) Int. Cl. G01N 33/53; G01N 33/566

(52) U.S. Cl. 435/7.1; 436/501

(57) ABSTRACT

Multiplex assays that allow for the detection and quantification of Rheumatoid Factor (RF) and anti-cyclic citrullinated peptide (CCP) antibodies in a single reaction mixture are provided.

The present patent application claims benefit of priority to U.S. Provisional Patent Application No. 61/090,082, filed, Aug. 19, 2008, which is incorporated by reference in its entirety for all purposes.

### BACKGROUND OF THE INVENTION

One of the most common autoimmune diseases is rheumatoid arthritis which affects 0.5-1% of the population. This systemic disease is marked by chronic inflammation of synovial joints which leads to destruction of cartilage and bone, and eventually to disability of the patient. Rheumatoid arthritis can severely affect the quality of life of a patient although it is not life threatening. Diagnosis of rheumatoid arthritis is based mostly on clinical observations but specific serum protein and serological tests are increasingly used to assist in the diagnosis. Several biomarkers are frequently used by rheumatologists including rheumatoid factor, anti-cyclic citrullinated peptide, and C reactive protein.

Rheumatoid factor (RF) is a term used to describe a group of autoantibodies known as rheumatoid factors. The RF test is considered the basic screen and hallmark for the autoimmune disorder rheumatoid arthritis (RA). RF is considered an early marker since its presence is associated with an increased risk of developing RA in people with mild arthritic symptoms.

RF factors include three subclasses that react with the crystallizable fragment (Fc fragment) of immunoglobulin G (IgG). Immunoglobulins are proteins normally found in the body that are used to produce antibodies. RF targets IgG proteins by combining with them to form deposits that lodge in the joints and tissues.

RF is not only present in patients with rheumatoid arthritis. It may also occur in patients with other autoimmune conditions such as systemic lupus erythematosus (SLE), Sjogren's syndrome, and occasionally scleroderma and polymyositis. It is also seen in the rheumatoid arthritis overlap syndromes, such as RA/SLE overlap and Scleroderma/RA overlap. The RF test may also be positive in other conditions and in the absence of disease, especially with advancing age. Other conditions that may cause a positive RF test result include chronic active hepatitis, sarcoidosis, chronic infection, various cancers, and syphilis.

The three subclasses of RF include IgM, IgA and IgG autoantibodies. Many tests for RF separately measure each of these subtypes. Each subtype is associated with a different symptom or disease process, and the simultaneous presence of all three types is usually only seen in rheumatoid arthritis. RF type IgA antibodies are associated with symptoms other than those affecting joints, such as mucosal and secretory problems and bone erosions. In patients with rheumatoid arthritis, IgM RF predominates and the other subtypes are usually present in lower amounts.

Rheumatoid factors are found in 50-90 percent of patients with classic RA, with higher concentrations seen in active disease. Higher titers are also seen in more severe forms of the disease. RF are also seen in 75-95 percent of patients with Sjogren's syndrome, 50-60 percent of patients with mixed connective tissue disease (MCTD), 25-40 percent of patients with the kidney disease IgA nephropathy, 15-35 percent of patients with SLE, 20-30 percent of patients with systemic sclerosis and 5-10 percent of patients with polymyositis and dermatomyositis. See, e.g., Moore, E., Rheumatoid Arthritis—Diagnosing Rheumatoid Arthritis and Related Disorders, March, 2006 (available on the internet at http address: //autoimmunedisease.suite101.con/article.cfm/rheumatoidfactortests).

The disease process in rheumatoid arthritis involves the presence of deposits of RF complexed or linked with IgG occurring in various tissues, such as the synovium or joints. These complexes interfere with the normal function of the joint and promote inflammation. The inflammation and the effects of immune system chemicals result in tissue damage and sometimes damage to blood vessels in the affected area. The RF test is used to diagnose arthritic conditions and to monitor response to treatment and prognosis. During periods of remission or a favorable response to treatment, RF titers fall and the RF test results may be negative.

Autoantibodies directed against citrullinated proteins (e.g., anti-CCP [cyclic citrullinated peptide] antibodies) are specific serological markers for rheumatoid arthritis. Anti-CCP antibodies may be detected in roughly 50-60% of patients with early rheumatoid arthritis at ‘baseline’ (at their initial encounter with a specialist, usually after 3-6 months of symptoms). See, e.g., Nell V, et al. Arthritis Res Ther. 5(Suppl 1):16 (2003). The specificity of anti-CCP is around 95-98% as regards undifferentiated forms of arthritis that do not develop into RA. IgM RF are often found in the same patients, but with much lower specificity for RA. One study using an anti-CCP assay showed a sensitivity of 55% and a specificity of 97% specificity for RA, when both anti-CCP and IgM RF were positive in the early stage of arthritis. See, e.g., Jansen A L, et al., J Rheumatol. 29:2074-6 (2002). Another study showed even higher prevalence at the first visit to clinics—anti-CCP antibodies were found in 70% of such patients. Interestingly, using stored samples, anti-CCP could be detected 1.5 to 9 years before the onset of arthritis. See, e.g., Rantapää-Dahlqvist S, et al., Arthritis Rheum. 48:2741-9 (2003). A study using an anti-CCP assay found progression from undifferentiated polyarthritis to RA in 93% of anti-CCP positive patients but only in 25% of anti-CCP negative patients after 3 years of follow-up. See, e.g., van Gaalen F A, et al., Arthritis Res Ther 5(suppl 1):28 (2003). Several observations have indicated that anti-CCP positive early RA patients may develop a more erosive disease than those without anti-CCP.

### BRIEF SUMMARY OF THE INVENTION

The present invention provides for methods for analyzing the level of rheumatoid factor (RF) and anti-cyclic citrullinated peptide (CCP) antibodies in a sample from a human.

In some embodiments, the method comprises
a) incubating the sample with a mixture of particles in a first suspension, said mixture of particles comprising:
   (i) a first capture agent immobilized on a first particle, said first capture agent capable of binding a human anti-CCP antibody; and
   (ii) a second capture agent immobilized on a second particle, said second capture agent capable of binding a human anti-RF antibody,
      wherein the incubation of step a) is performed under conditions to allow anti-CCP antibodies and RF antibodies, if present, to bind to the first capture agent and the second capture agent, respectively;
b) recovering said particles from said first suspension; and
c) incubating said recovered particles with a mixture of labeled binding members, the members comprising:
   (i) a labeled first detection agent that binds anti-CCP antibodies; and
   (ii) a labeled second detection agent that binds anti-RF antibodies,
      wherein the incubation of step c) is performed under conditions to allow the labeled first detection agent and the labeled second detection agent to bind the bound anti-CCP antibodies and the bound RF antibodies and, respectively, if present; and
d) recovering said particles from said second suspension; and
e) detecting the amount of label bound to said particles thus recovered from said second suspension, thereby obtaining values individually representative of the levels of rheumatoid factor and anti-cyclic citrullinated peptide.

In some embodiments, the first capture agent comprises a CCP; and the first detection agent comprises an antibody that binds to human IgG. In some embodiments, the incubating step a) comprises incubating an exogenous anti-CCP antibody that competes with an anti-CCP antibody in the sample, and the labeled first detection agent binds to the exogenous anti-CCP antibody but does not bind to the anti-CCP antibody in the sample.

In some embodiments, the first capture agent comprises an antibody that binds to human IgG; and the first detection agent comprises a CCP.

In some embodiments, the second capture agent comprises an Fc region of an antibody; and the second detection agent comprises an antibody having binding specificity for a human IgM, IgG, or IgA. In some embodiments, the incubating step a) comprises incubating an exogenous anti-FC antibody that competes with an anti-RF antibody in the sample, and the labeled first detection agent binds to the exogenous anti-FC antibody but does not bind to the anti-RF antibody in the sample.

In some embodiments, the second capture agent comprises an antibody having binding specificity for a human IgM, IgG, or IgA; and the second detection agent comprises an Fc region of an antibody.

In some embodiments, the method comprises
a) incubating the sample with a mixture of particles in a first suspension, said mixture of particles comprising:
   (i) a cyclic citrullinated peptide (CCP) immobilized on a first particle; and
   (ii) an antibody immobilized on a second particle, wherein the antibody has a binding specificity for human IgM, IgG or IgA,
      wherein the incubation of step a) is performed under conditions to allow anti-CCP antibodies and RF antibodies, if present in the sample, to bind to the CCP and the antibody, respectively;
b) recovering said particles from said first suspension; and
c) incubating said recovered particles with a mixture of labeled binding members, the members comprising:
   (i) a labeled polypeptide comprising an Fc region of an antibody; and
   (ii) a labeled antibody that binds human IgG,
      wherein the incubation of step c) is performed under conditions to allow the labeled polypeptide and the labeled antibody to bind the bound RF antibodies and the bound anti-CCP antibodies, respectively, if present; and
d) recovering said particles from said second suspension; and
e) detecting the amount of label bound to said particles thus recovered from said second suspension, thereby obtaining values individually representative of the levels of rheumatoid factor and anti-cyclic citrullinated peptide.

In some embodiments, the particles in section a) parts (i) and (ii) are distinguishable by a flow cytometry distinguishable characteristic that is independent of the CCP or antibody immobilized on the particles; and step d) comprises detecting the amount of label bound to said particles recovered from said second suspension while correlating by flow cytometry the amount of label thus detected to the particle to which label is bound.

In some embodiments, the sample is a blood or serum sample.

In some embodiments, step a) comprises incubating an antibody having binding specificity for a human IgM immobilized on the second particle. In some embodiments, step a) comprises incubating an antibody having binding specificity for a human IgG immobilized on the second particle. In some embodiments, step a) comprises incubating an antibody having binding specificity for a human IgA immobilized on the second particle. In some embodiments, step a) comprises incubating an antibody having binding specificity for a human IgG and an antibody having binding specificity for a human IgM immobilized on the second particle. In some embodiments, step a) comprises incubating an antibody having binding specificity for a human IgA and an antibody having binding specificity for a human IgM immobilized on the second particle.

In some embodiments, step a) comprises incubating an antibody having binding specificity for human IgG immobilized on the second particle and an antibody having binding specificity for a human IgM immobilized on the second particle. In some embodiments, step a) comprises incubating an antibody having binding specificity for human IgA immobilized on the second particle and an antibody having binding specificity for a human IgM immobilized on the second particle. In some embodiments, step a) comprises incubating an antibody having binding specificity for human IgA immobilized on the second particle and an antibody having binding specificity for a human IgG immobilized on the second particle. In some embodiments, step a) comprises incubating an antibody having binding specificity for human IgA immobilized on the second particle, an antibody having binding specificity for a human IgM immobilized on the second particle, and an antibody having binding specificity for human IgG immobilized on the second particle. In some embodiments, step a) comprises incubating a first antibody having binding specificity for one of the group consisting of human IgA, IgG and IgM immobilized on the second particle and a second antibody having binding specificity for one of the group consisting of human IgA, IgG and IgM immobilized on a third particle, wherein the second antibody binds to a different member of the group than the first antibody. In some embodiments, step a) comprises incubating a first antibody having binding specificity for one of the group consisting of human IgA, IgG and IgM immobilized on the second particle, a second antibody having binding specificity for one of the group consisting of human IgA, IgG and IgM immobilized on a third particle, and a second antibody having binding specificity for one of the group consisting of human IgA, IgG and IgM immobilized on a third particle, wherein each of the first, second and third antibodies bind to a different member of the group.

In some embodiments, the particles are magnetically-responsive particles and steps b) and d) comprise subjecting the first and second suspensions, respectively, to a magnetic field to cause the particles to adhere to a reaction vessel wall.

In some embodiments, the labels are fluorescent labels.

In some embodiments, the first and second particles comprise different dyes such that the first and second particles are distinguishable by flow cytometry, and wherein step d) comprises distinguishing the first and second particles by flow cytometry.

In some embodiments, the particles in the mixture are approximately the same diameter. In some embodiments, the first and second particles have sufficiently different diameter to be distinguishable by flow cytometry.

In some embodiments, said Fc region is a non-human Fc region and/or said antibody immobilized on said second particle is a non-human antibody and/or said labeled antibody that binds human IgG is a non-human antibody. In some embodiments, said Fc region is a horse Fc region and/or said antibody immobilized on said second particle is a donkey antibody and/or said labeled antibody that binds human IgG is a mouse antibody.

The present invention also provides kits for detecting factors associated with rheumatoid arthritis. In some embodiments, the kit comprises:

a) a cyclic citrullinated peptide (CCP) immobilized on a first solid support; and

b) an antibody immobilized on a second solid support, wherein the antibody has a binding specificity for human IgM, IgG or IgA.

In some embodiments, the solid supports of a) and b) are in the same reaction vessel. In some embodiments, the solid supports in a) and b) are particles. In some embodiments, the solid supports in a) and b) are different particles.

In some embodiments, the particles are magnetically-responsive particles.

In some embodiments, the antibody immobilized on the second solid support is a non-human antibody.

In some embodiments, the kit comprises an antibody having binding specificity for human IgG immobilized on the second particle and an antibody having binding specificity for a human IgM immobilized on the second particle. In some embodiments, the kit comprises an antibody having binding specificity for human IgA immobilized on the second particle and an antibody having binding specificity for a human IgM immobilized on the second particle. In some embodiments, the kit comprises an antibody having binding specificity for human IgA immobilized on the second particle and an antibody having binding specificity for a human IgG immobilized on the second particle. In some embodiments, the kit comprises an antibody having binding specificity for human IgA immobilized on the second particle, an antibody having binding specificity for a human IgM immobilized on the second particle, and an antibody having binding specificity for human IgG immobilized on the second particle. In some embodiments, the kit comprises a first antibody having binding specificity for one of the group consisting of human IgA, IgG and IgM immobilized on the second particle and a second antibody having binding specificity for one of the group consisting of human IgA, IgG and IgM immobilized on a third particle, wherein the second antibody binds to a different member of the group than the first antibody. In some embodiments, the kit comprises a first antibody having binding specificity for one of the group consisting of human IgA, IgG and IgM immobilized on the second particle, a second antibody having binding specificity for one of the group consisting of human IgA, IgG and IgM immobilized on a third particle, and a second antibody having binding specificity for one of the group consisting of human IgA, IgG and IgM immobilized on a third particle, wherein each of the first, second and third antibodies bind to a different member of the group.

In some embodiments, the method further comprises,

c) a labeled antibody that binds human IgG; and/or

d) a labeled polypeptide comprising an Fc region of an antibody.

In some embodiments, the labels are fluorescent labels.

In some embodiments, the first and second solid supports comprise different dyes such that the first and second solid supports are distinguishable by flow cytometry.

In some embodiments, the particles in the mixture are approximately the same diameter. In some embodiments, the first and second particles have sufficiently different diameter to be distinguishable by flow cytometry.

In some embodiments, said Fc region is a non-human Fc region and/or said labeled antibody that binds human IgG is a non-human antibody. In some embodiments, said Fc region is a horse Fc region and/or said antibody immobilized on said second particle is a donkey antibody and/or said labeled antibody that binds human IgG is a mouse antibody.

In some embodiments, the kit further comprises a control comprising an amount of anti-CCP antibodies and/or an amount of RF.

The present invention also provides for reaction mixtures comprising

a) a cyclic citrullinated peptide (CCP) immobilized on a first solid support; and

b) an antibody immobilized on a second solid support, wherein the antibody has a binding specificity for human IgM, IgG or IgA.

In some embodiments, the reaction mixture further comprises a biological sample.

In some embodiments, the solid supports in a) and b) are particles. In some embodiments, the solid supports in a) and b) are different particles. In some embodiments, the particles are magnetically-responsive particles.

In some embodiments, the antibody immobilized on the second solid support is a non-human antibody.

In some embodiments, the reaction mixture further comprises,

c) a labeled antibody that binds human IgG; and/or

d) a labeled polypeptide comprising an Fc region of an antibody.

In some embodiments, the labels are fluorescent labels.

In some embodiments, the reaction mixture comprises an antibody having binding specificity for human IgG immobilized on the second particle and an antibody having binding specificity for a human IgM immobilized on the second particle. In some embodiments, the reaction mixture comprises an antibody having binding specificity for human IgA immobilized on the second particle and an antibody having binding specificity for a human IgM immobilized on the second particle. In some embodiments, the reaction mixture comprises an antibody having binding specificity for human IgA immobilized on the second particle and an antibody having binding specificity for a human IgG immobilized on the second particle. In some embodiments, the reaction mixture comprises an antibody having binding specificity for human IgA immobilized on the second particle, an antibody having binding specificity for a human IgM immobilized on the second particle, and an antibody having binding specificity for human IgG immobilized on the second particle. In some embodiments, the reaction mixture comprises a first antibody having binding specificity for one of the group consisting of human IgA, IgG and IgM immobilized on the second particle and a second antibody having binding specificity for one of the group consisting of human IgA, IgG and IgM immobilized on a third particle, wherein the second antibody binds to a different member of the group than the first antibody. In some embodiments, the reaction mixture comprises a first antibody having binding specificity one of the group consisting of human IgA, IgG and IgM immobilized on the second particle, a second antibody having binding specificity for one of the group consisting of human IgA, IgG and IgM immobilized on a third particle, and a second antibody having binding specificity for one of the group consisting of human IgA, IgG and IgM immobilized on a third particle, wherein each of the first, second and third antibodies bind to a different member of the group.

In some embodiments, the first and second solid supports comprise different dyes such that the first and second particles are distinguishable by flow cytometry.

In some embodiments, the particles in the mixture are approximately the same diameter. In some embodiments, the first and second particles have sufficiently different diameter to be distinguishable by flow cytometry.

In some embodiments, said Fc region is a non-human Fc region and/or said labeled antibody that binds human IgG is a non-human antibody. In some embodiments, said Fc region is a horse Fc region and/or said antibody immobilized on said second particle is a donkey antibody and/or said labeled antibody that binds human IgG is a mouse antibody.

### DEFINITIONS

Cyclic citrullinated peptide” or “CCP” refers to citrullinated peptides or proteins that can act as epitopes bound by autoantibodies in the sera of rheumatoid arthritis patients. See, e.g., WO/2007/039280; WO 98/08946; WO 98/22503; WO 99/28344; WO 99/35167, WO 01/46222, WO 03/050542; and Schellekens, G. A. et al., Arthritis Rheum. 43:155-163 (2000).

“Anti-CCP antibodies” refers to autoantibodies that specifically bind to CCP. The accumulation of anti-CCP antibodies is associated with the development and occurrence of rheumatoid arthritis.

“Rheumatoid factor” or “RF” refers to an autoantibody that binds to the Fc region of IgG antibodies. See, e.g., WO/2005/085859.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into, e.g., antibodies and/or other proteins at any position. Any method known in the art for conjugating the antibody to the label may be employed, e.g., using methods described in Hermanson, Bioconjugate Techniques 1996, Academic Press, Inc., San Diego. Alternatively, methods using high affinity interactions may achieve the same results where one of a pair of binding partners binds to the other, e.g., biotin, streptavidin. The proteins of the invention as described herein can be directly labeled as with isotopes, chromophores, lumiphores, chromogens, or indirectly labeled such as with biotin to which a streptavidin complex (optionally including, e.g., a fluorescent, radioactive, or other moiety that can be directly detected) may later bind. Thus, a biotinylated antibody is considered a “labeled antibody” as used herein.

“Antibody” refers to a polypeptide encoded by an immunoglobulin gene or immunoglobulin genes, or fragments thereof, which specifically bind and recognize an analyte (antigen). The recognized immunoglobulin light chains are classified as either kappa or lambda. Immunoglobulin heavy chains are classified as gamma, mu, alpha, delta, or epsilon, which in turn define the immunoglobulin classes, IgG, IgM, IgA, IgD and IgE, respectively.

An exemplary immunoglobulin G (IgG antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody near the disulfide linkages in the hinge region to produce F(ab′)₂, a dimer of Fab which itself is a light chain joined to V_{H}-C_{H}1 by a disulfide bond. The F(ab′)₂ may be reduced under mild conditions to break the disulfide linkage in the hinge region, thereby converting the F(ab′)₂ dimer into two Fab′ monomers. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term “antibody,” as used herein, also includes antibody fragments either produced by the modification of whole antibodies or those synthesized de novo using recombinant DNA methodologies (e.g., single chain Fv).

The phrase “specifically (or selectively) binds to an antibody” or “specifically (or selectively) immunoreactive with” or “having binding specificity for”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein can be selected to obtain antibodies specifically immunoreactive with that protein and not with other proteins. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

The term “biological sample” encompasses a variety of sample types obtained from an organism. The term encompasses bodily fluids such as blood, saliva, serum, plasma, urine and other liquid samples of biological origin, solid tissue samples, such as a biopsy specimen or tissue cultures or cells derived therefrom and the progeny thereof. As described herein, typically, the biological sample will be a bodily fluid or tissue that contains detectable amounts of antibodies. The term encompasses samples that have been manipulated in any way after their procurement, such as by treatment with reagents, solubilization, sedimentation, or enrichment for certain components. The term encompasses a clinical sample, and also includes cells in cell culture, cell supernatants, cell lysates, serum, plasma, other biological fluids, and tissue samples.

“Particles” and “microparticles” are used synonymously and interchangeably in the present invention. Particles are spherical or near-spherical polymerized structures that serve as a solid support in the immunoassays of the present invention.

“Multiplex” assays are analyses that simultaneously measure the levels of more than one analyte in a single sample.

A “biologic” as used in the present invention is any product such as an antibody or other protein derived from a living organism.

An “endogenous” biologic as used in the present invention is a biochemical agent found in the human sample that is being assayed.

An “exogenous” biologic as used in the present invention is a biochemical agent not found in the human sample that is being assayed.

### BRIEF DESCRIPTION OF THE DRAWINGS

FIG. 1 is a diagram of a first assay format in accordance with the present invention, showing a CCP-bound particle, binding members, and types of binding reactions used to analyze the level of anti-CCP in a human sample.

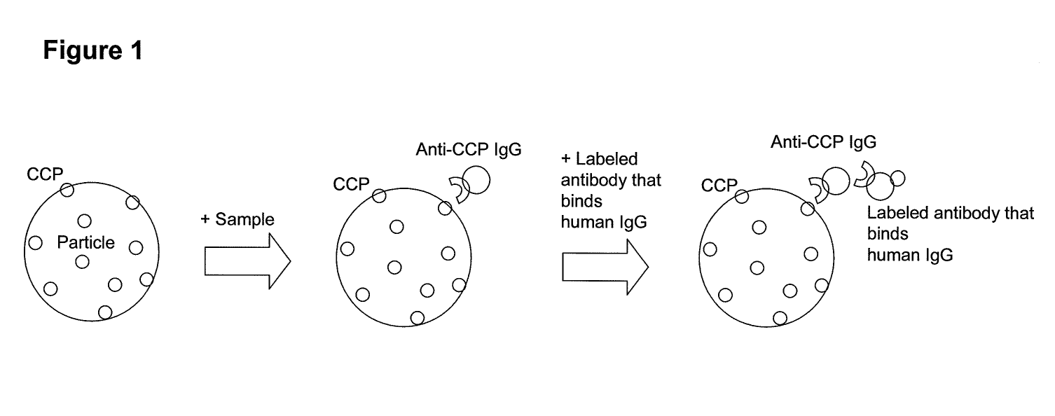

FIG. 2 is a diagram of an assay format using a biotin:streptavidin system in accordance with the present invention, showing a CCP-bound particle, binding members, and types of binding reactions used to analyze the level of anti-CCP in a human sample.

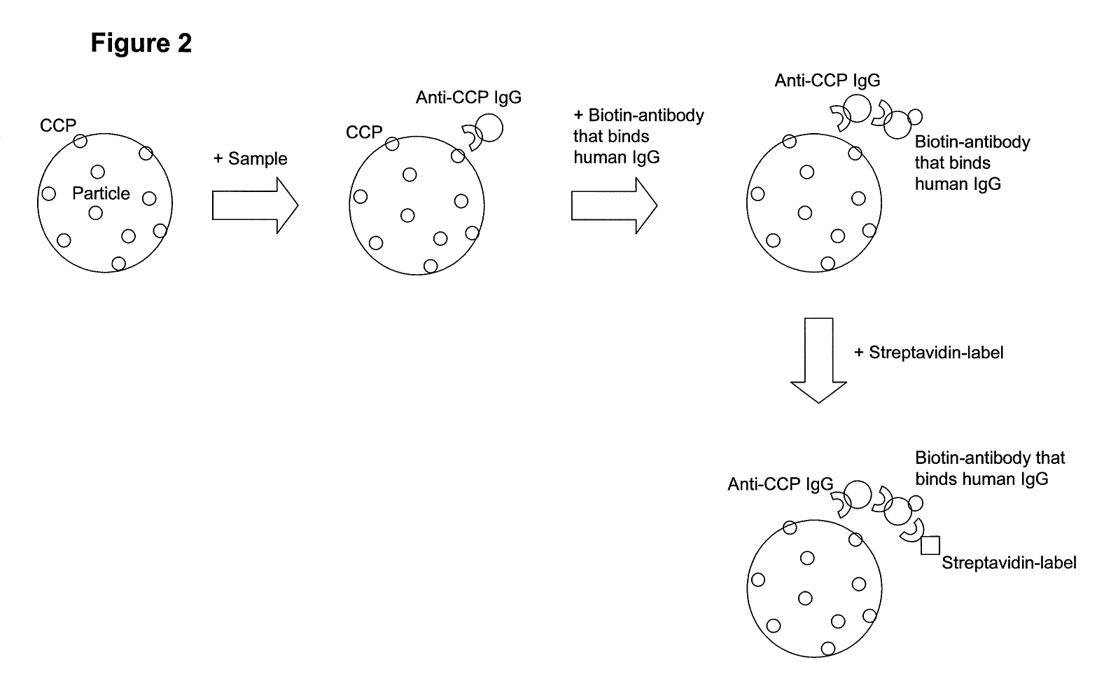

FIG. 3 is a diagram of an assay format using a hapten:anti-hapten system in accordance with the present invention, showing a CCP-bound particle, binding members, and types of binding reactions used to analyze the level of anti-CCP in a human sample.

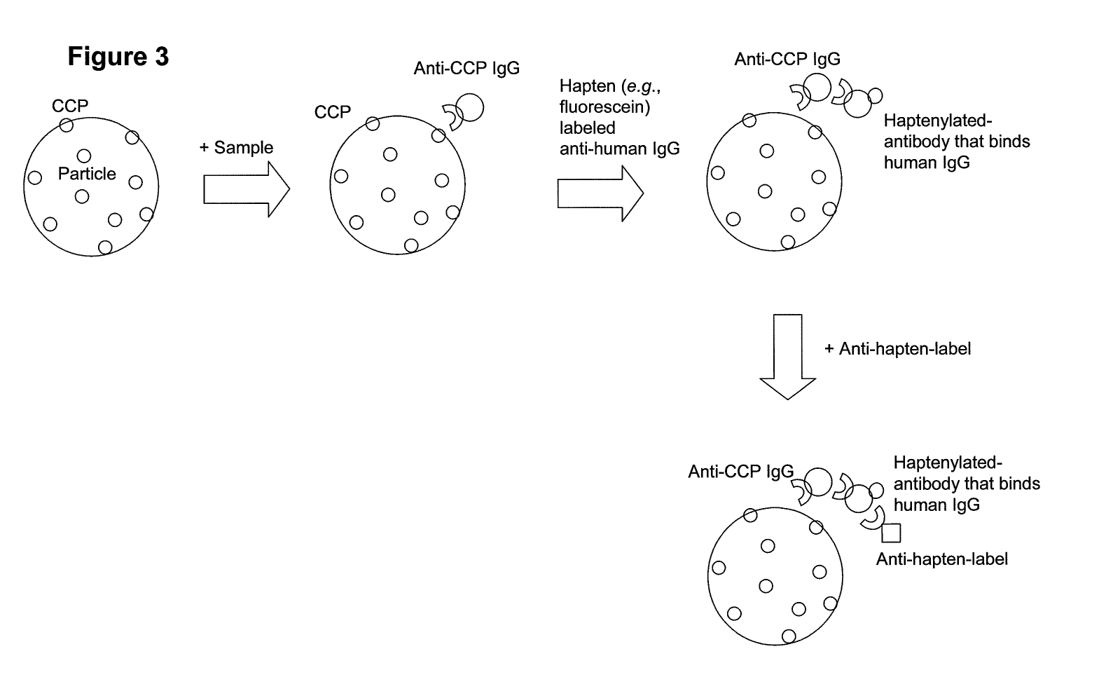

FIG. 4 is a diagram of an assay format (an IgG capture assay) in accordance with the present invention, showing an anti-human IgG-bound particle, binding members, and types of binding reactions used to analyze the level of anti-CCP in a human sample.

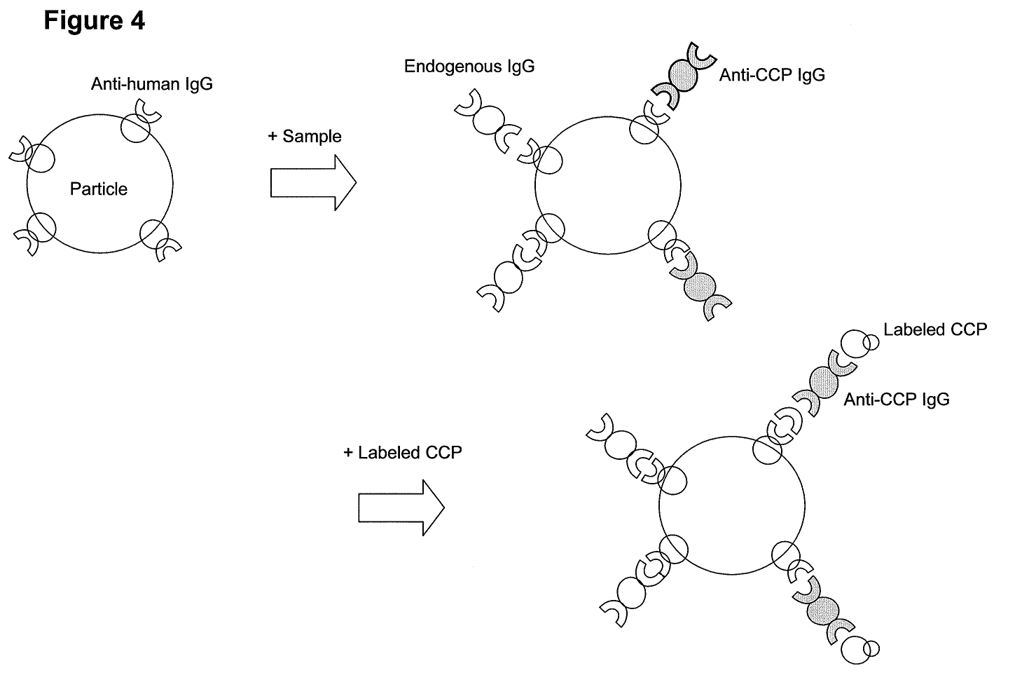

FIG. 5 is a diagram of an assay format (a competitive assay) in accordance with the present invention, showing a CCP-bound particle, binding members, and types of binding reactions used to analyze the level of anti-CCP in a human sample.

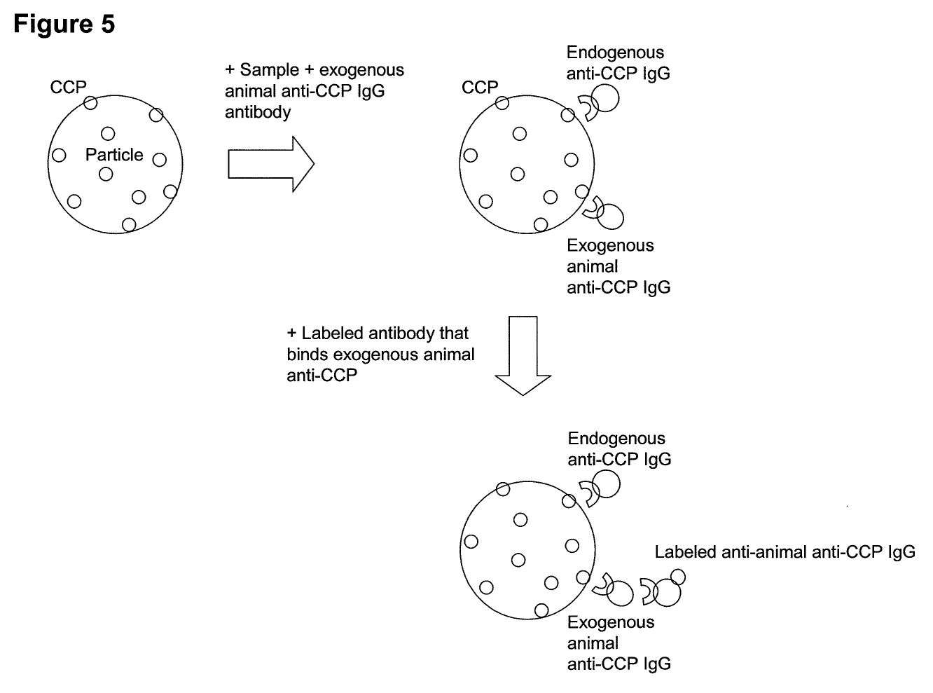

FIG. 6 is a diagram of an assay format in accordance with the present invention, showing an anti-human IgM bound particle, binding members, and types of binding reactions used to analyze the level of IgM RF in a human sample. As a first subtype of this assay format, the anti-human IgM may be a specific antibody for whole human IgM. As a second subtype of this assay format, the anti-human IgM may be a mu-chain specific antibody thus providing a mu-capture assay.

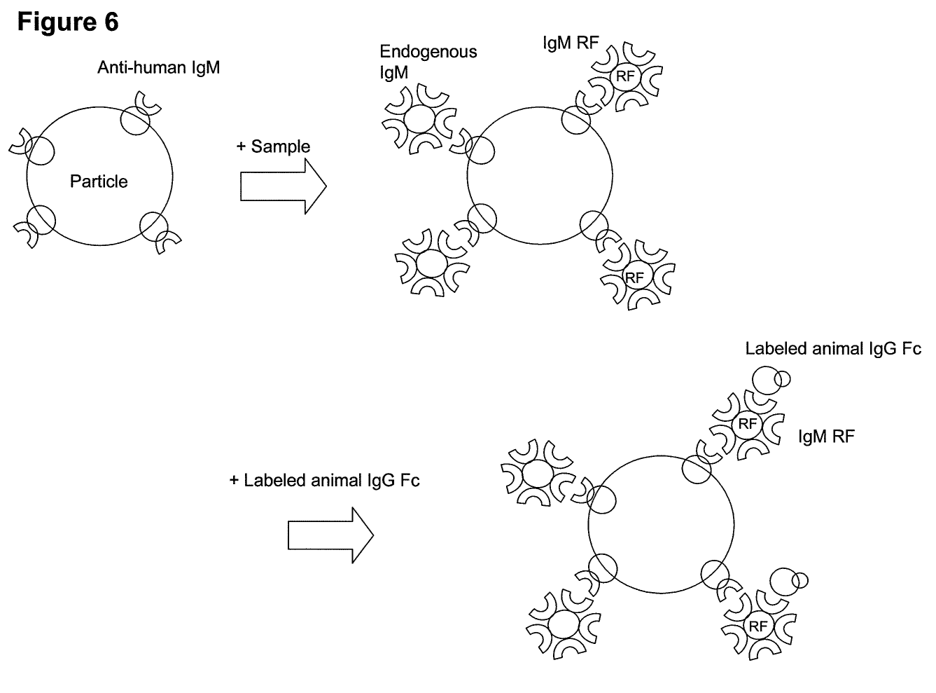

FIG. 7 is a diagram of an assay format using a biotin:streptavidin system in accordance with the present invention, showing an anti-human IgM bound particle, binding members, and types of binding reactions used to analyze the level of IgM RF in a human sample.

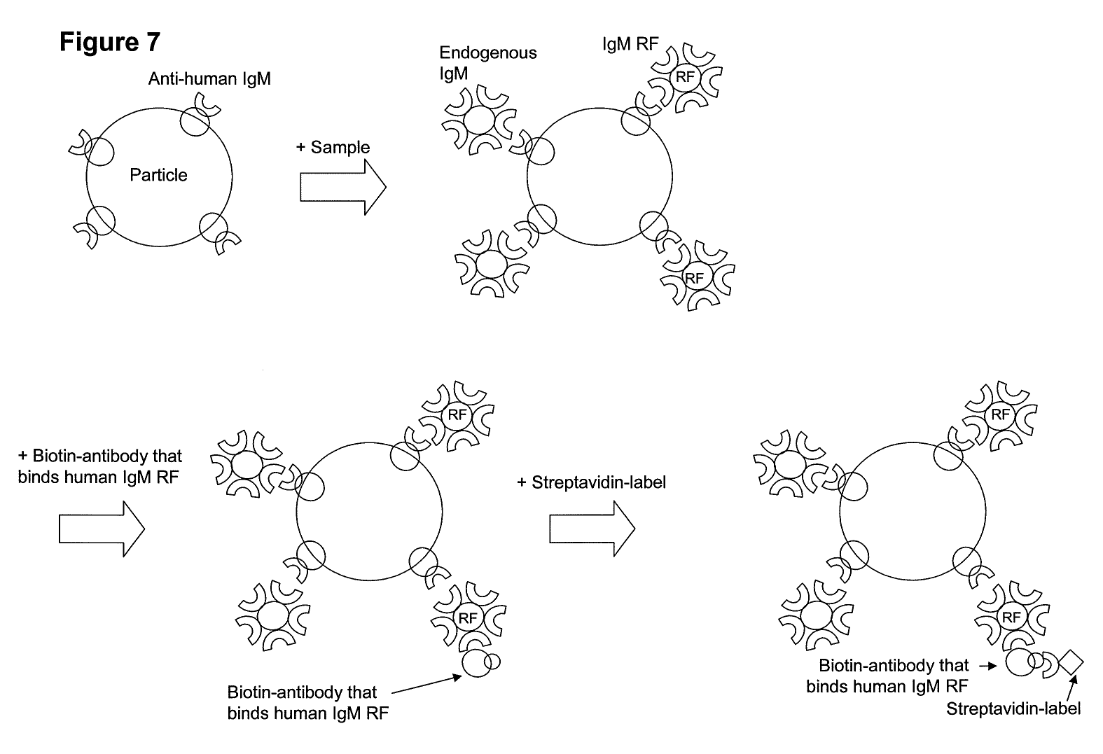

FIG. 8 is a diagram of an assay format in accordance with the present invention, showing a human or animal IgG or IgG Fc bound particle, binding members, and types of binding reactions used to analyze the level of IgM RF in a human sample. This assay format is the reverse of that depicted in FIG. 6 in that the solid-support biologic and labeled biologic of FIG. 6 are reversed in this assay format. This reverse assay can also be constructed for IgM RF or IgA RF with use of labeled anti-human IgM or labeled anti-human IgA, respectively.

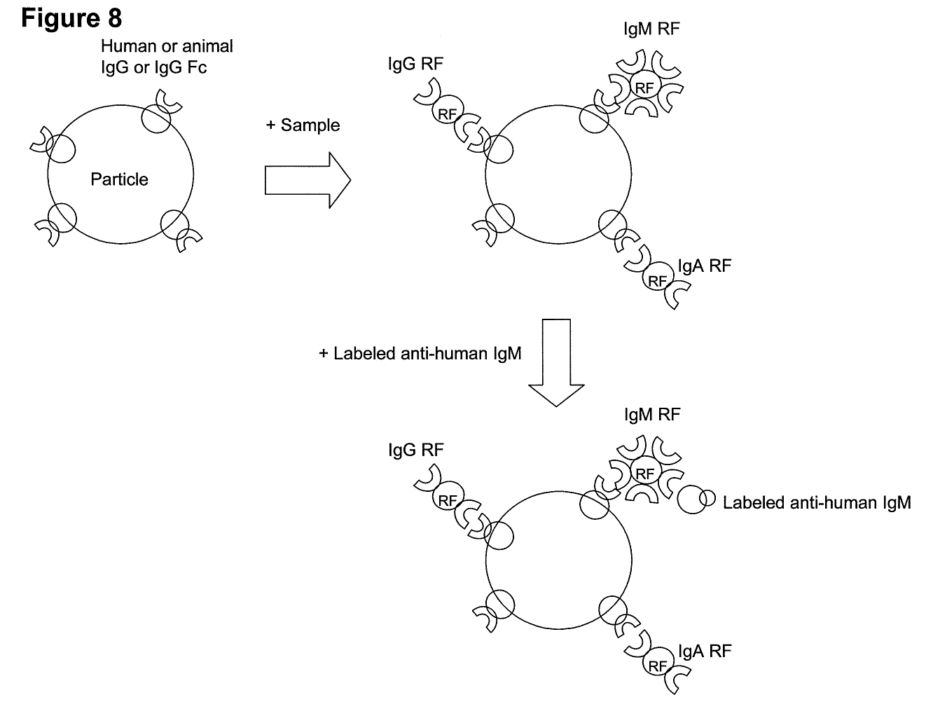

FIG. 9 is a diagram of an assay format in accordance with the present invention, showing an anti-human IgA bound particle, binding members, and types of binding reactions used to analyze the level of IgA RF in a human sample. As a first subtype of this assay format, the anti-human IgA may be a specific antibody for whole human IgA. As a second subtype of this assay format, the anti-human IgA may be an alpha-chain specific antibody thus providing an alpha-capture assay.

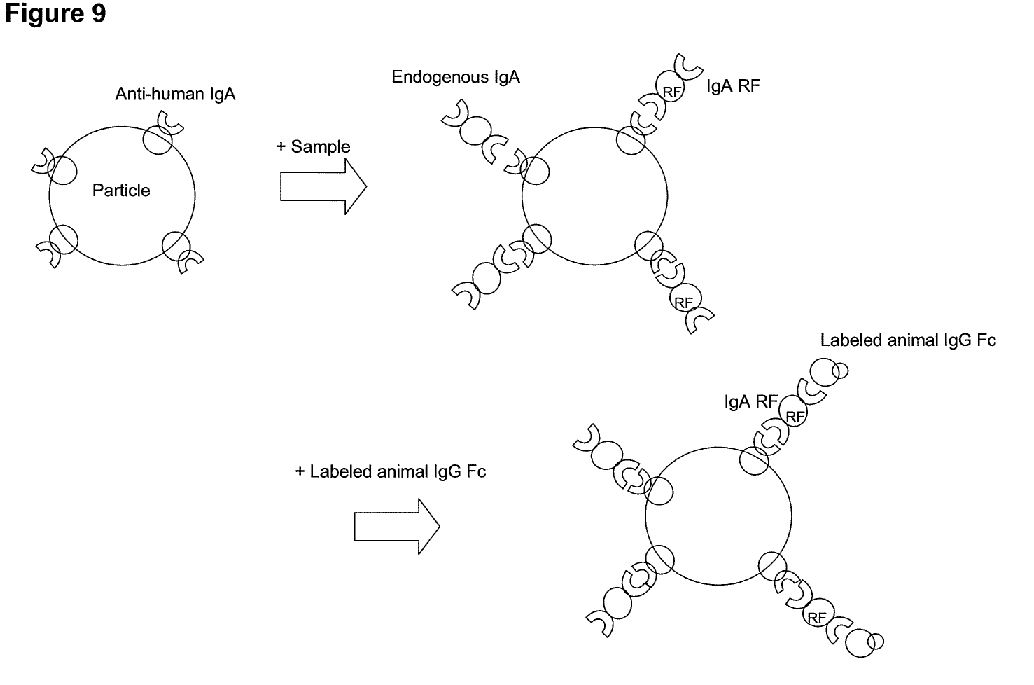

FIG. 10 is a diagram of an assay format in accordance with the present invention, showing an anti-human IgG bound particle, binding members, and types of binding reactions used to analyze the level of IgG RF in a human sample. As a first subtype of this assay format, the anti-human IgG may be a specific antibody for whole human IgG. As a second subtype of this assay format, the anti-human IgG may be a gamma-chain specific antibody thus providing a gamma-capture assay.

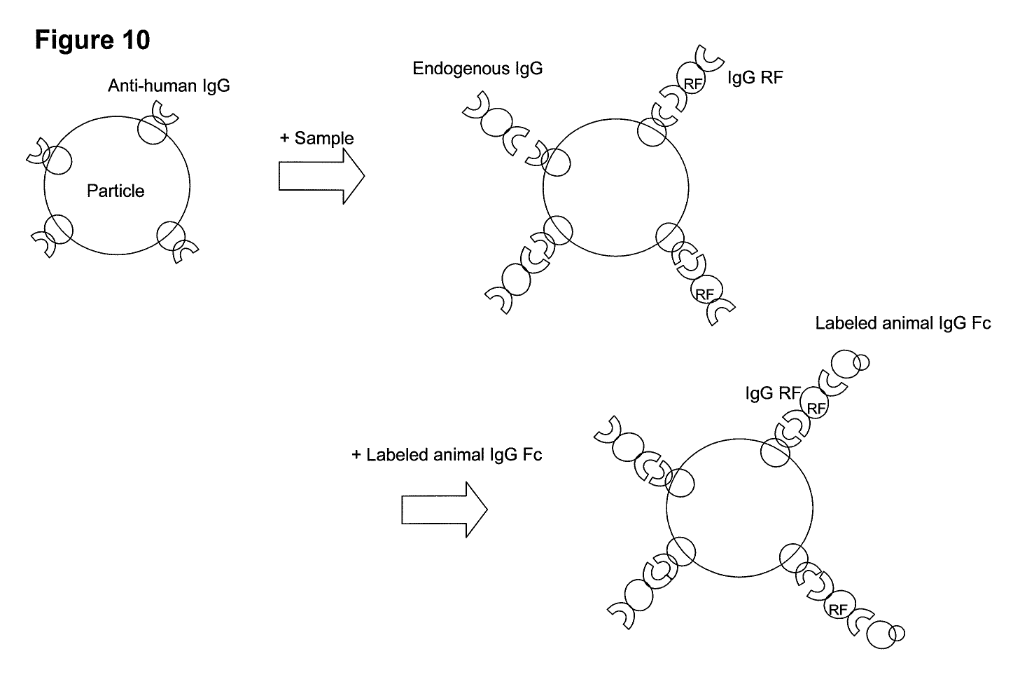

FIG. 11 is a diagram of an assay format in accordance with the present invention, showing a particle co-bound with anti-human IgM and anti-human IgA, binding members, and types of binding reactions used to analyze the sum level of IgM RF and IgA RF in a human sample.

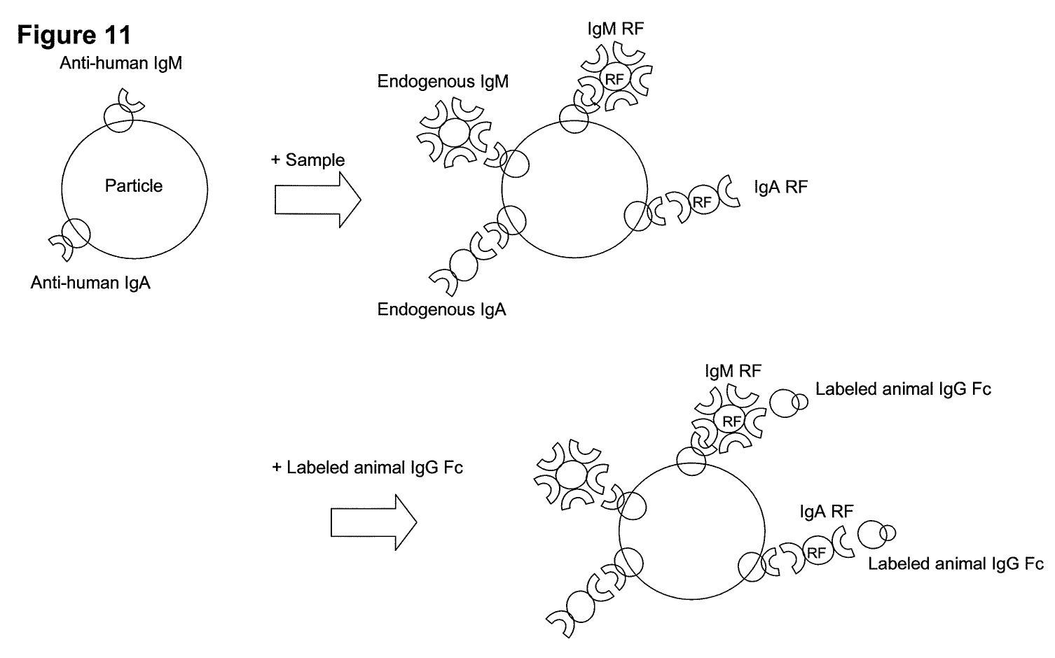

FIG. 12 is a diagram of an assay format in accordance with the present invention, showing a particle co-bound with anti-human IgM and anti-human IgG, binding members, and types of binding reactions used to analyze the sum level of IgM RF and IgG RF in a human sample.

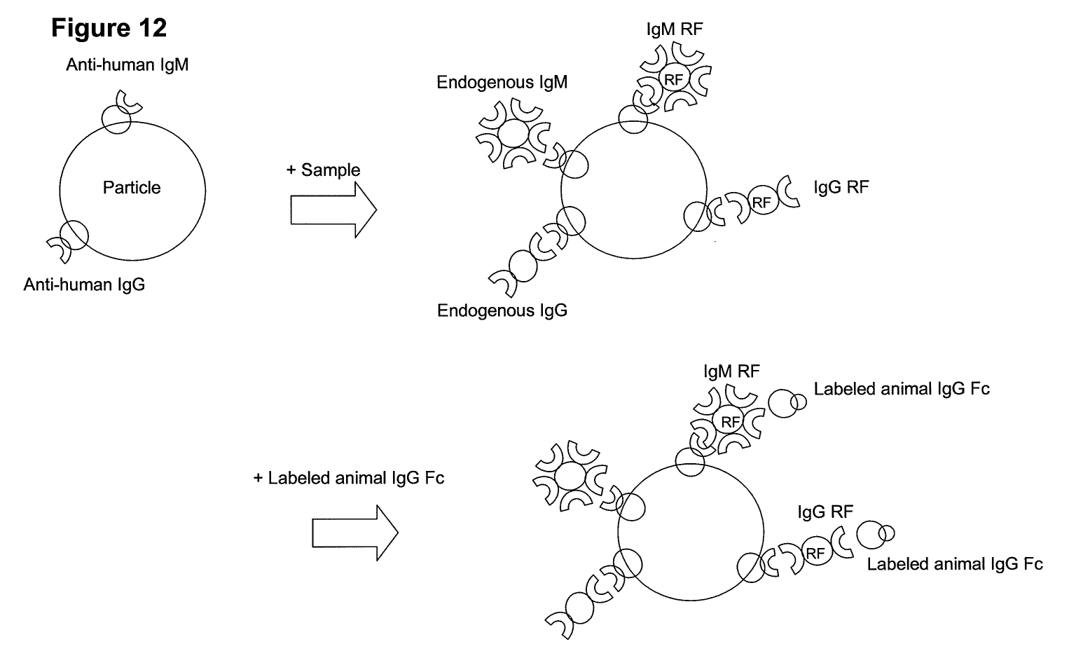

FIG. 13 is a diagram of an assay format in accordance with the present invention, showing a particle co-bound with anti-human IgG and anti-human IgA, binding members, and types of binding reactions used to analyze the sum level of IgG RF and IgA RF in a human sample.

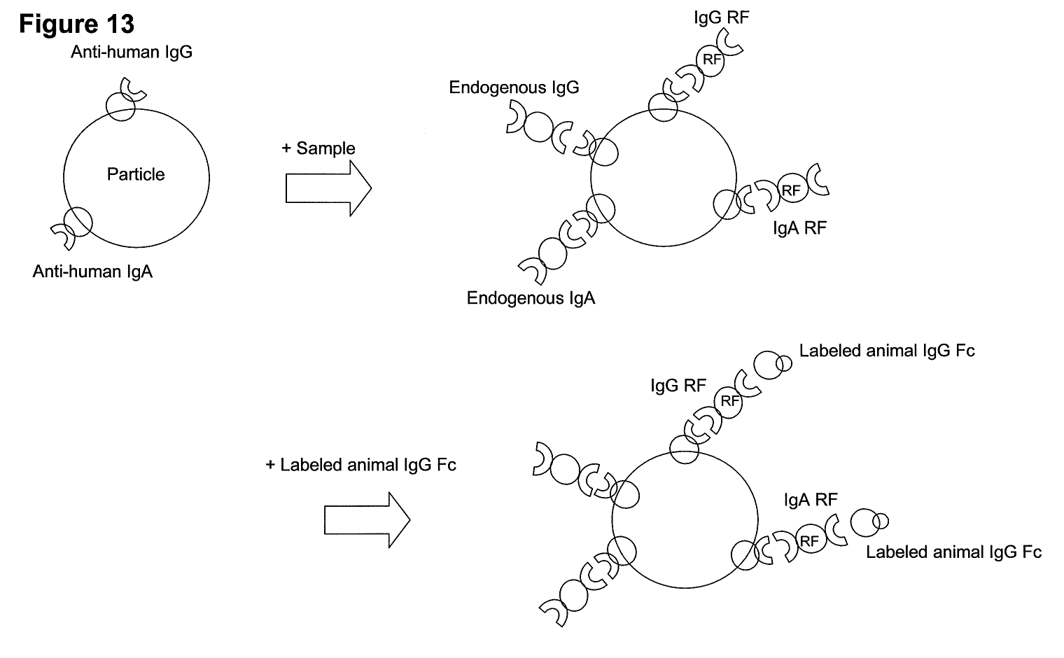

FIG. 14 is a diagram of an assay format in accordance with the present invention, showing a particle co-bound with anti-human IgM, anti-human IgA, and anti-human IgG binding members, and types of binding reactions used to analyze the sum level of IgM RF, IgA RF, and IgG RF in a human sample.

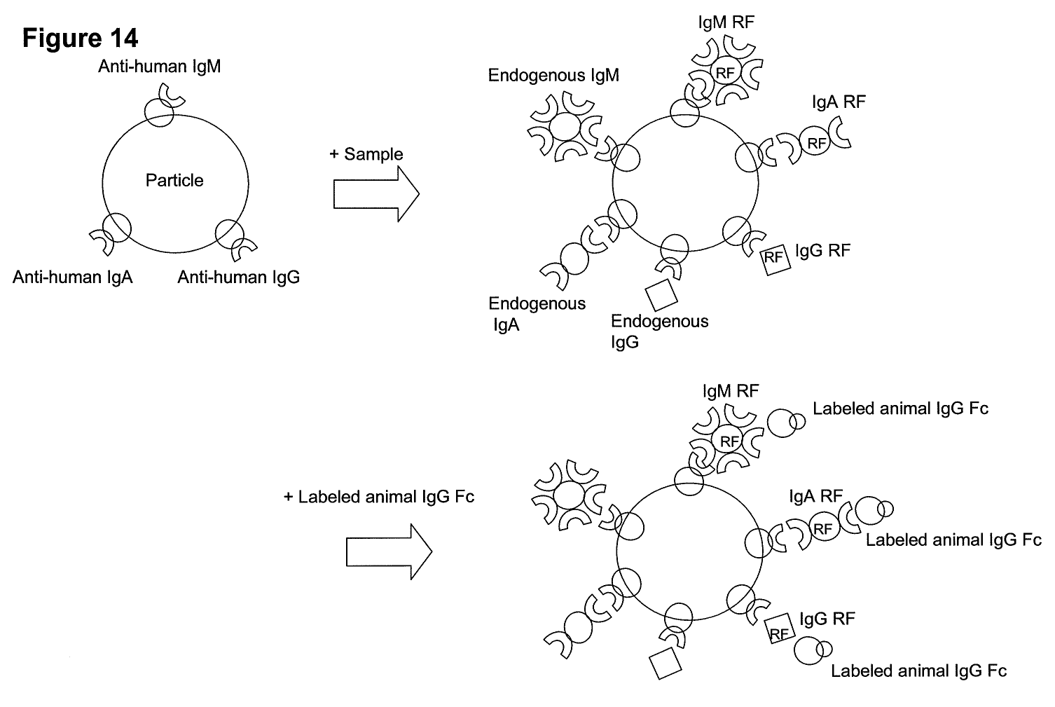

FIG. 15 is a diagram of an assay format (a competitive assay) in accordance with the present invention, showing an IgG Fc bound particle, binding members, and types of binding reactions used to analyze the sum level of IgM RF, IgA RF, and IgG RF in a human sample.

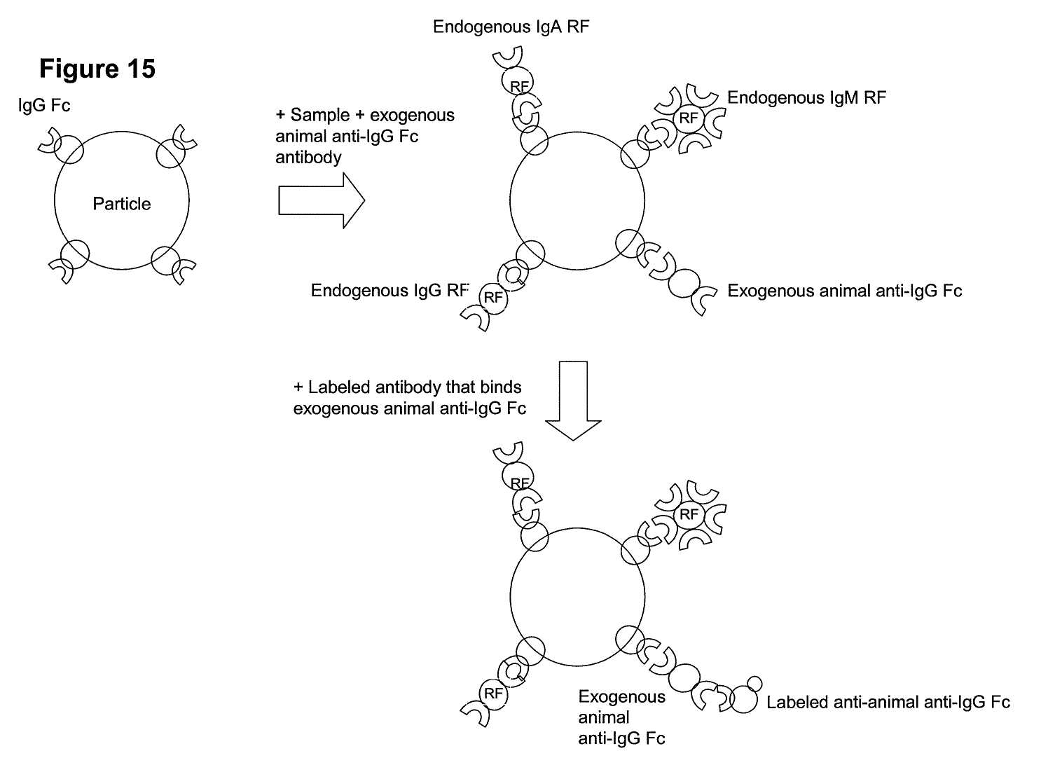

FIG. 16 is a diagram of a multiplex assay format in accordance with the present invention, showing two discrete particles, the first particle bound with anti-human IgM and the second particle bound with anti-human IgG, binding members, and types of binding reactions used to simultaneously but separately analyze the level of IgM RF and IgG RF in a human sample.

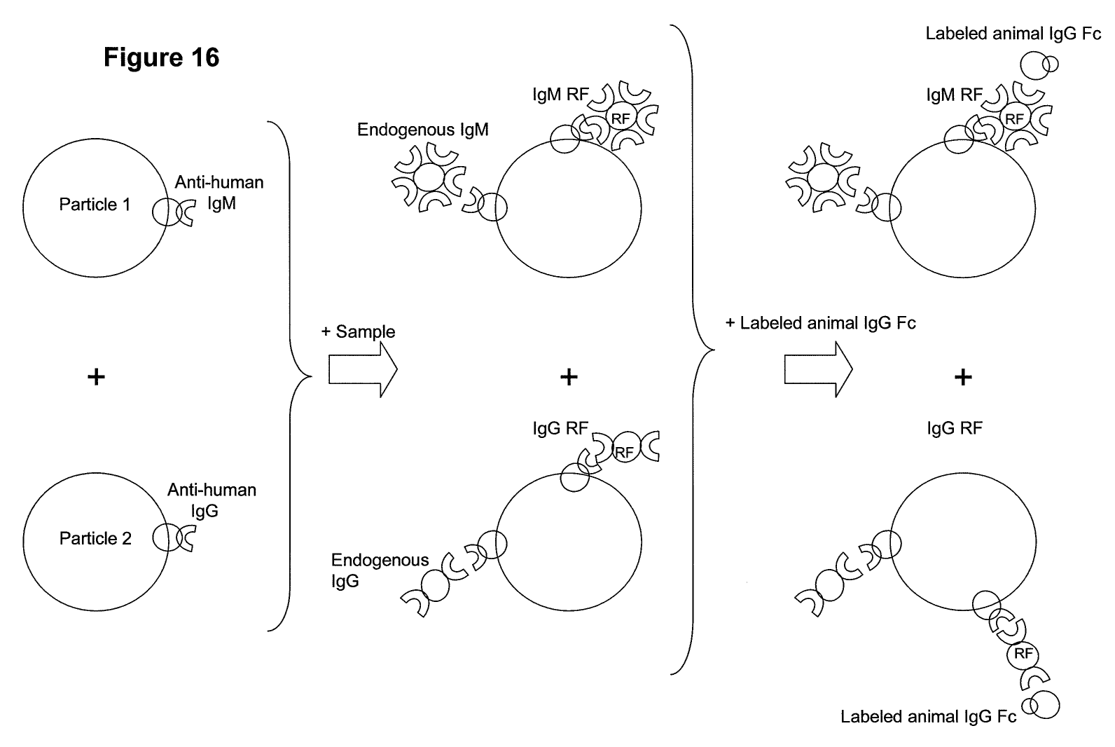

FIG. 17 is a diagram of a multiplex assay format in accordance with the present invention, showing two discrete particles, the first particle bound with anti-human IgM and the second particle bound with anti-human IgA, binding members, and types of binding reactions used to simultaneously but separately analyze the level of IgM RF and IgA RF in a human sample.

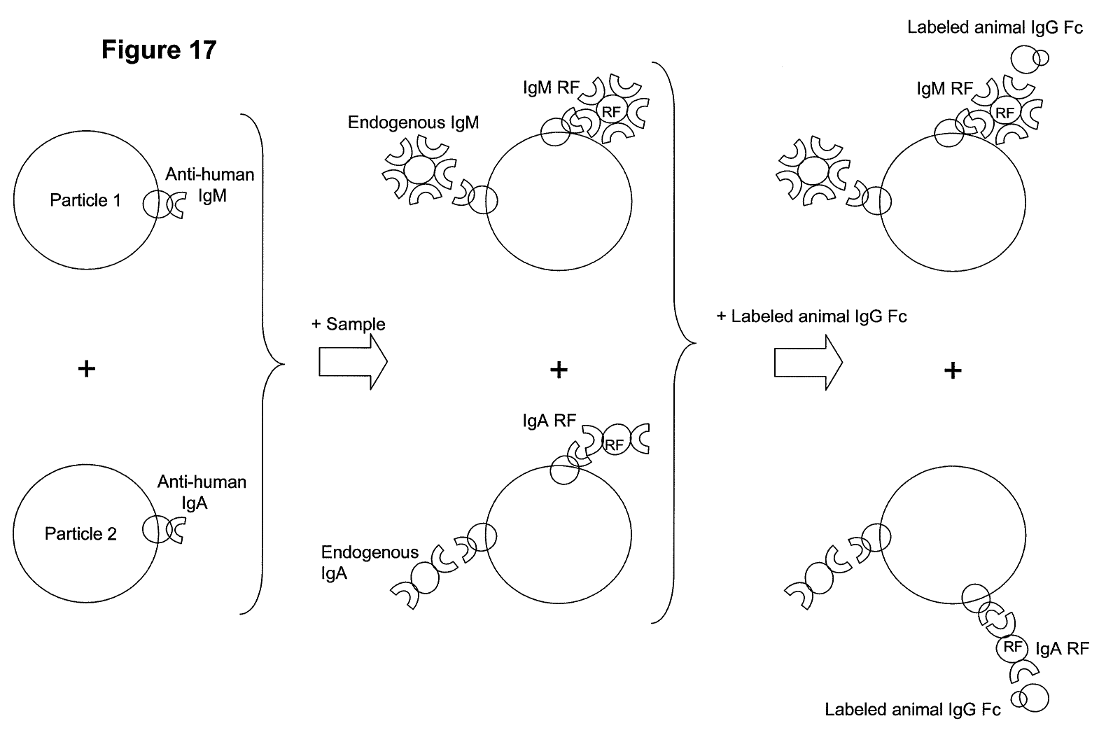

FIG. 18 is a diagram of a multiplex assay format in accordance with the present invention, showing two discrete particles, the first particle bound with anti-human IgA and the second particle bound with anti-human IgG, binding members, and types of binding reactions used to simultaneously but separately analyze the level of IgA RF and IgG RF in a human sample.

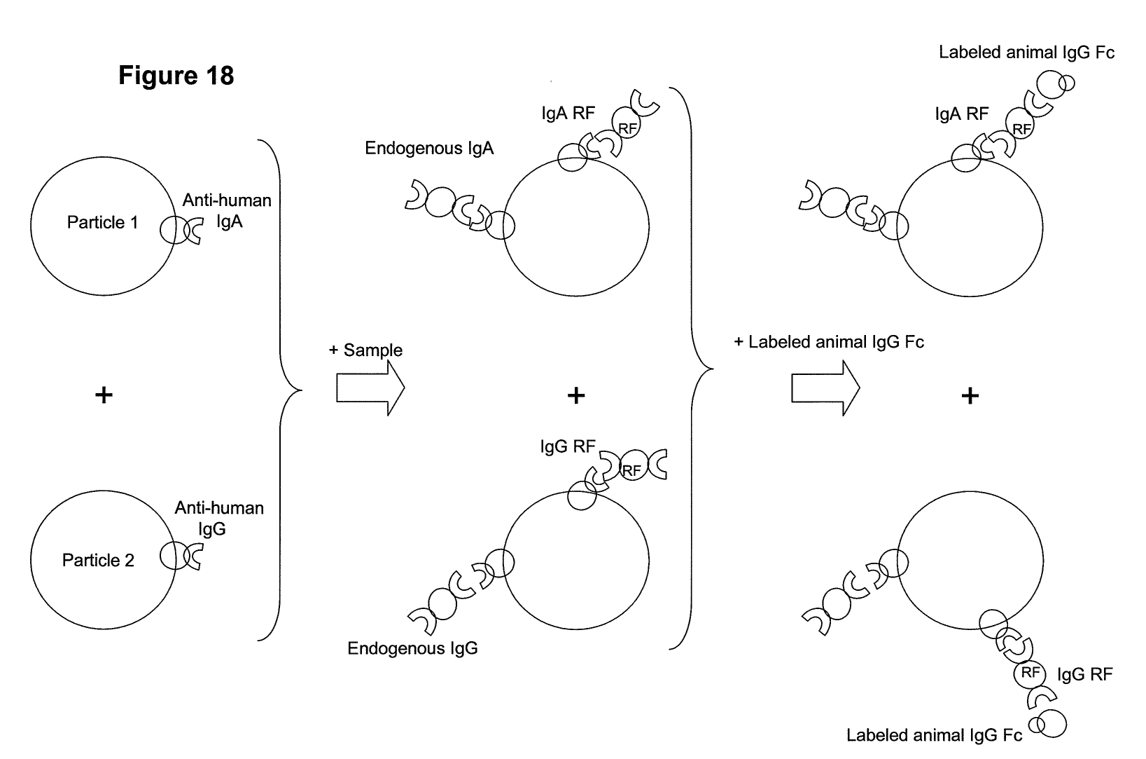

FIG. 19 is a diagram of a multiplex assay format in accordance with the present invention, showing three discrete particles, the first particle bound with anti-human IgM, the second particle bound with anti-human IgG, and the third particle bound with anti-human IgA, binding members, and types of binding reactions used to simultaneously but separately analyze the level of IgM RF, IgG RF, and IgA RF in a human sample.

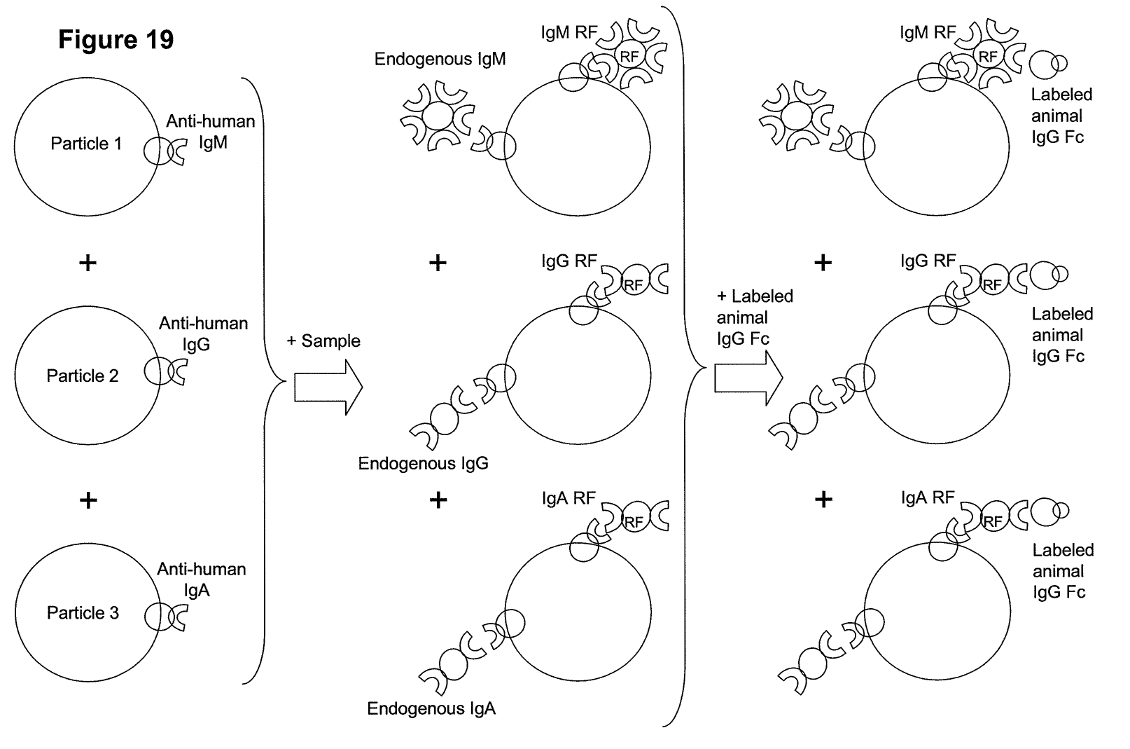

FIG. 20 is a diagram of a multiplex assay format in accordance with the present invention, showing two discrete particles, the first particle bound with CCP and the second particle bound with anti-human IgM, binding members, and types of binding reactions used to simultaneously but separately analyze the level of anti-CCP and IgM RF in a human sample. This assay format can be used in an analogous manner to assay for CCP and IgG RF, CCP and IgA RF, and CCP and any two or all three of IgM RF, IgG RF, and IgA RF.

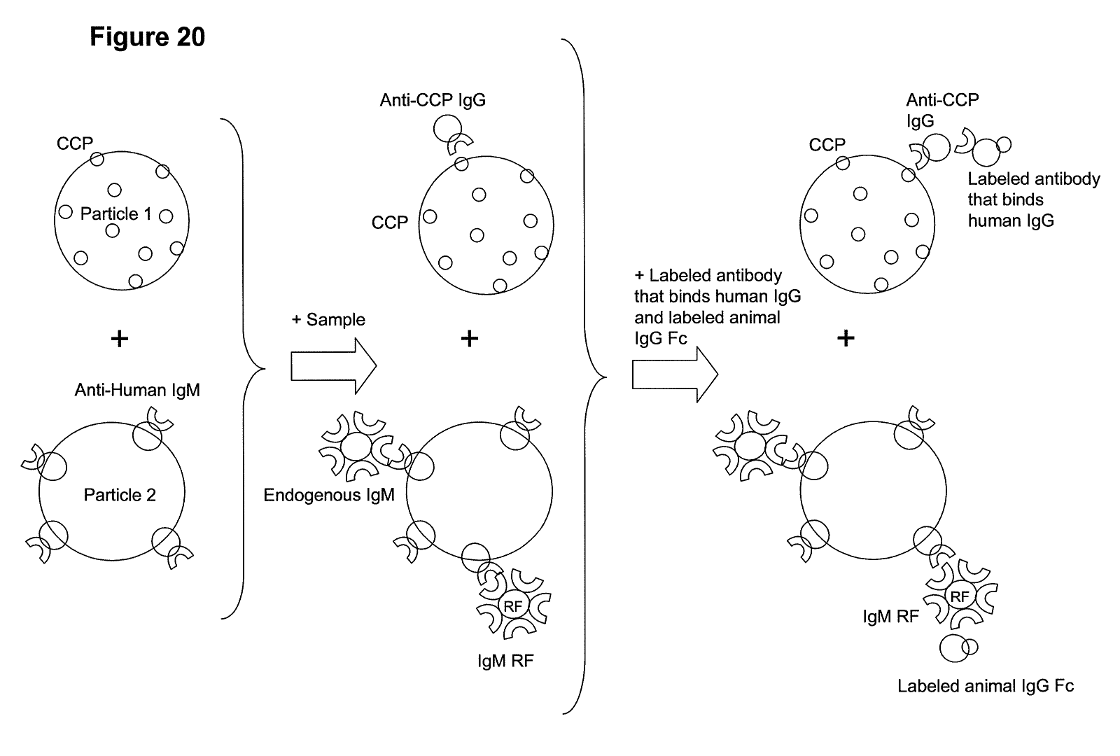

FIG. 21 is a calibration curve for anti-CCP generated from a multiplexed assay for anti-CCP and RF IgM in accordance with the present invention.

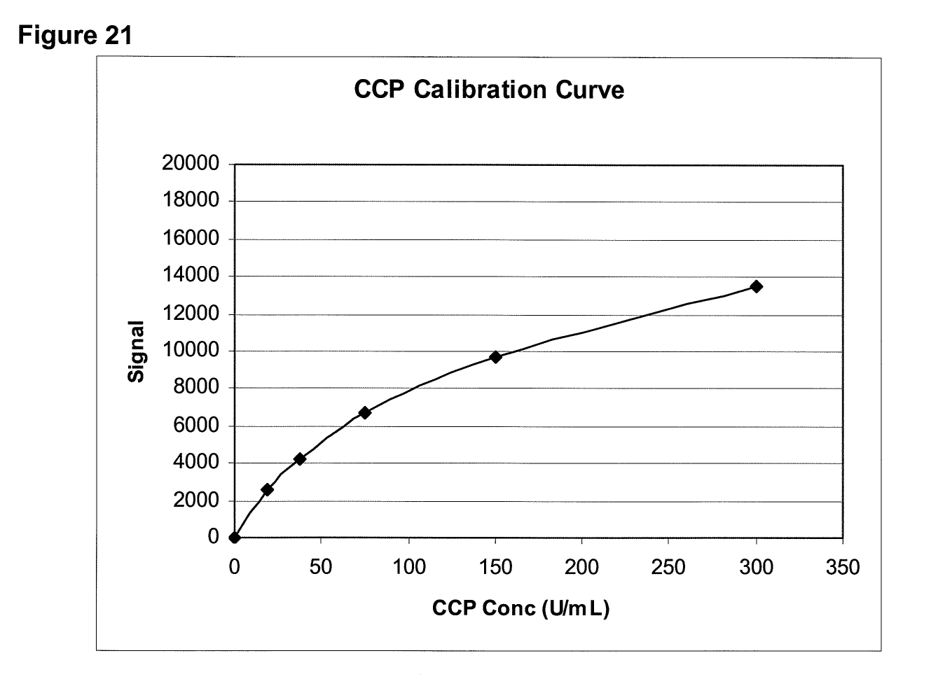

FIG. 22 is a calibration curve for RF IgM generated from a multiplexed assay for anti-CCP and RF IgM in accordance with the present invention.

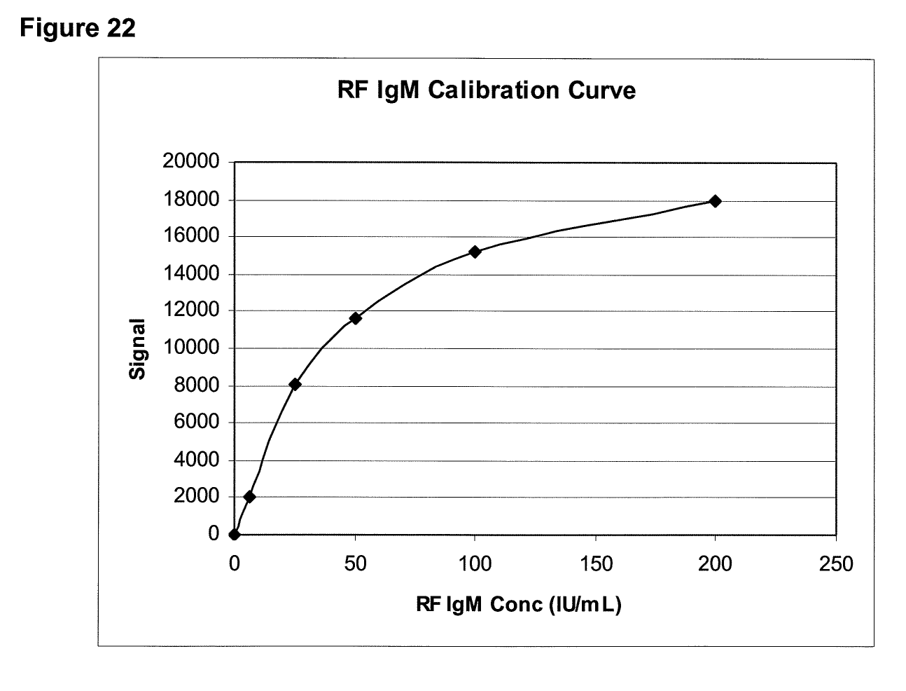

FIG. 23 is a calibration curve for RF Total generated from a multiplexed assay for anti-CCP, RF IgM and RF Total in accordance with the present invention.

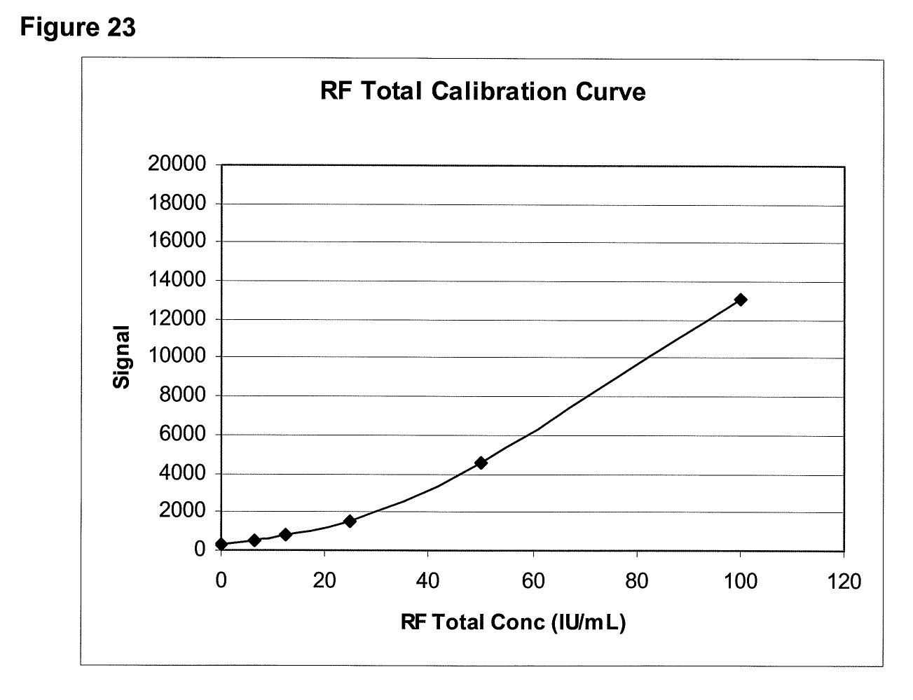

### DETAILED DESCRIPTION

### I. Introduction

The present invention provides for efficient detection of both anti-cyclic citrullinated peptide (CCP) antibodies and rheumatoid factor (RF) in one reaction mixture. A sample can be incubated with a first capture agent immobilized on a first solid support, the first capture agent capable of binding a human anti-CCP antibody and a second capture agent immobilized on a second solid support, the second capture agent capable of binding a human anti-RF antibody immobilized on a second particle followed by an incubating step with a labeled first detection agent that binds anti-CCP antibodies and a labeled second detection agent that binds anti-RF antibodies.

Simultaneous detection of anti-CCP antibodies and RF can be achieved, for example, by incubating a sample from a human with CCP immobilized on a first particle and an antibody having binding specificity for human IgM, IgG or IgA on a second particle, where the first and second particles are distinguishable. The incubated particles are subsequently recovered from the incubation mixture and then incubated with labeled binding members, where a first labeled binding member is a labeled peptide comprising the Fc region of an antibody and a second labeled binding member is a labeled antibody that binds human IgG. Binding of the first labeled binding member to the first particle indicates the presence and/or amount of anti-CCP antibodies in the sample and binding of the second labeled binding member to the second particle indicates the presence and/or amount of RF in the sample. The methods of the present invention are thus advantageous in part because they allow for efficient detection of anti-CCP antibodies and RF in a single reaction mixture.

### II. Methods of Detection

Detection of anti-CCP antibodies and RF can be achieved by first capturing anti-CCP antibodies and RF, if present in a sample, onto separate, distinguishable solid supports, and then detecting the captured anti-CCP antibodies and RF using labeled reagents that detect anti-CCP antibodies and RF. This is achieved by selection of reagents for capture and detection of anti-CCP antibodies that do not cross react with the reagents for capture and detection of RF, thereby allowing for detection of both anti-CCP antibodies and RF in a single reaction mixture.

A. General Approach

i. Detection of CCP

A number of formats for detection of anti-CCP antibodies are provided in the figures. Generally, a first capture agent, immobilized on a first solid support, is used to capture the anti-CCP antibodies. The first capture agent can be, for example, CCP or an antibody that binds human IgG. Once captured, the anti-CCP antibody can be detected using a labeled detection agent. The detection agent can be, for example, which ever of CCP or an antibody that binds human IgG was not used in the capture step.

In some embodiments, anti-CCP antibodies in a sample are initially captured by contacting the sample with CCP immobilized on first solid support under conditions to allow for binding of the anti-CCP antibodies, if present in the sample, to the immobilized CCP. The presence of the captured anti-CCP antibodies is then detected.

In some embodiments of the invention, the CCP is immobilized onto a first particle. CCP can be purchased commercially, e.g., in a biotinylated form from Axis-Shield (Norton, Mass.). Many CCPs are described in, e.g., US Patent Publication Nos. 2007/0087380 and 2002/0143143. Once anti-CCP antibodies in the sample are captured, the particles are recovered and separated from the remaining reagents in the mixture. For example, in some embodiments, the sample is removed from the particles by washing the particles in an appropriately buffered solution. Particles can be recovered by any method known in the art. In some cases, the particles are pelleted by centrifugation and the remaining sample (i.e., the supernatant) is removed from the particles. In some embodiments, the particles are responsive to a magnetic field and a magnetic field is applied such that the liquid in a sample is removed while the particles adhere a reaction vessel wall, separating the remaining liquid from the particles. The particles can optionally be washed, e.g., one or more times with an appropriate buffer, if desired.

The captured anti-CCP antibodies are subsequently detected and optionally quantified. In embodiments where the capture step is specific for anti-CCP antibodies (and thus does not significantly capture other IgG antibodies), the anti-CCP antibodies can be detected by incubating the captured CCP with a labeled antibody that specifically binds to human IgG, thereby allowing the labeled antibody to bind to the captured anti-CCP antibodies. Excess labeled antibody is subsequently removed, and the remaining labeled antibody (associated with the first particles) is detected and optionally quantified. The presence and quantity of the label can be used to estimate the amount of anti-CCP antibodies in the original sample, for example, by comparing the quantity of label to a calibration curve based on known amounts of anti-CCP antibodies as is well known in the art.

Any type of anti-human IgG antibody can be used in the assay for detection of anti-CCP antibodies. Anti-human antibodies can be generated by administering human IgG, optionally with an adjuvant, to a non-human animal thereby stimulating production of antibodies in the animal that bind to human IgG. Optionally, anti-human IgG antibodies can be generated in vitro, e.g., by screening phage display antibody libraries or other antibody libraries. The anti-human IgG antibodies can be for example, mouse, rat, rabbit, goat, donkey or other non-human animal antibodies.

Alternatively, a competition assay can be used to detect the anti-CCP antibodies in the sample. In these embodiments, CCP immobilized on a solid support (e.g., a particle) is incubated with a sample as well as an exogenous anti-CCP antibody, thus allowing for competition of the exogenous anti-CCP antibody with any endogenous anti-CCP antibody in the sample. The amount of exogenous anti-CCP antibody used is sufficient to bind to most or all of the binding sites on the solid support but is not in such high quantity to prevent detection of competition from endogenous anti-CCP antibody. A labeled detection agent (e.g., an antibody that binds IgG) is then used that binds to the exogenous anti-CCP antibody but not the endogenous anti-CCP antibody from the sample. Differential recognition can be achieved, for example, by using a non-human antibody as the exogenous antibody and using a detection agent that recognizes that non-human antibody but does not recognize a corresponding human antibody. Reduction in signal from the label associated with the solid support is thus related to increased amount of endogenous anti-CCP antibody in the sample. In a further alternative, the exogenous anti-CCP antibody can be directly or indirectly labeled, thereby optionally removing the need for a separate detection agent.

ii. Detection of RF

A number of formats for detection of RF molecules (i.e., antibodies that bind the Fc region of human IgG) are provided in the figures. Generally, a first capture agent, immobilized on a first solid support, is used to capture the RF. The first capture agent can be, for example, (1) an antibody with binding specificity for human IgM, IgG, or IgA, or (1) an Fc region of an antibody. Once captured, the RF can be detected using a labeled detection agent. The detection agent can be, for example, which ever of (1) an antibody with binding specificity for human IgM, IgG, or IgA, or (2) an Fc region of an antibody was not used in the capture step.

In some embodiments, RF in a sample are initially captured by contacting the sample with a capture antibody that specifically binds at least one of human IgM, IgG or IgA that is immobilized onto a second solid support (a solid support different and distinguishable from the first solid support used to detect anti-CCP antibodies, discussed above). The contacting step is performed under conditions to allow for binding of the RF, if present in the sample, to the immobilized capture antibody. The presence of the captured RF is then detected. In some embodiments of the invention, the capture antibody is immobilized onto a “second” particle distinguishable from the first particle discussed above in reference to anti-CCP antibody detection.

As discussed above, the capture antibody binds at least one of human IgM, IgG or IgA. Thus, in some embodiments, the capture antibody binds to human IgM. In some embodiments, the capture antibody binds to human IgG. In some embodiments, the capture antibody binds to human IgA. If one desires to detect a combination of two or more of IgM, IgG, or IgA RF, then a combination of two or more of the above-described capture antibodies are used, as appropriate. Depending on what RF information is desired, the combination of two or more capture antibodies can be linked to the same particles (i.e., the “second particles”), or each different capture antibody type can be immobilized on a different particle (i.e., a “third particle”, and if necessary, a “fourth particle”) that is distinguishable from the first and second (and as appropriate, the third) particles.

Once the RF in the sample is captured, the particles are recovered and separated from the remaining reagents in the mixture. For example, in some embodiments, the sample is removed from the particles by washing the particles in an appropriately buffered solution. Particles can be recovered by any method known in the art. In some cases, the particles are pelleted by centrifugation and the remaining sample (i.e., the supernatant) is removed from the particles. In some embodiments, the particles are responsive to a magnetic field and a magnetic field is applied such that the liquid in a sample is removed while the particles adhere a reaction vessel wall, separating the remaining liquid from the particles. The particles can optionally be washed, e.g., one or more times with an appropriate buffer, if desired.

In some embodiments, the initial capture of RF is not specific. The immobilized capture antibody captures any IgM, IgG, and/or IgA (depending on the specificity of the capture antibody) present in the sample. Therefore, the detection regent used to detect the presence of the captured RF is specific for RF. The detection reagent used can be, for example, a labeled polypeptide comprising an Fc region of a non-human antibody. While RF binds to human Fc, because of similarity of Fc regions between species, non-human Fc regions can also be used if desired, so long as the Fc regions bind to RF in human samples. For example, in a non-limiting example, the inventors have found that goat or horse Fc regions are non-human Fc regions that effectively bind to RF.

Excess labeled detection reagent is subsequently removed, and the remaining labeled detection (associated with the second, and optionally, third and fourth, particles) is detected and optionally quantified. The presence and quantity of the label can be used to estimate the amount of RF in the original sample, for example, by comparing the quantity of label to a calibration curve based on known amounts of RF as is well known in the art.

Alternatively, a competition assay can be used to detect the RF in the sample. In these embodiments, a polypeptide comprising an Fc region of an antibody, immobilized on a solid support (e.g., a particle), is incubated with a sample as well as an exogenous anti-Fc antibody, thus allowing for competition of the exogenous anti-Fc antibody with any endogenous RF in the sample. The amount of exogenous anti-Fc antibody used is sufficient to bind to most or all of the binding sites on the solid support but is not in such high quantity to prevent detection of competition from endogenous RF. A labeled detection agent (e.g., an antibody having binding specificity for human IgM, IgG, or IgA) is then used that binds to the exogenous anti-Fc antibody but not the endogenous RF from the sample. Reduction in signal from the label associated with the solid support is thus related to increased amount of endogenous RF in the sample. In a further alternative, the exogenous anti-Fc antibody can be directly or indirectly labeled, thereby optionally removing the need for a separate detection agent.

B. Combination of Anti-CCP and RF Assays in a Single Reaction Mixture

Because the above described assays for anti-CCP antibodies and for RF do not include reagents that cross-react with each other, any of the two assays (anti-CCP and RF) described above can be efficiently combined into a single reaction mixture. Thus, in some embodiments, the methods of the invention comprise a single reaction vessel comprising at least a first and second particle, wherein the first particle includes an immobilized CCP and the second particle includes an immobilized capture antibody that has specificity for human IgM, IgG, or IgA. A sample is added to the vessel and incubated under conditions to allow the anti-CCP antibodies and/or RF present in the sample to bind to the immobilized CCP and RF capture antibody, respectively. The particles are separated from the remaining sample, for example, by washing the particles with a buffer that does not interfere with the binding of the anti-CCP antibodies and RF to the CCP and antibodies, respectively, immobilized on the particles. The particles are subsequently separated from the wash buffer and contacted with a labeled polypeptide comprising an Fc region of an antibody and a labeled antibody that binds human IgG under conditions such that the labeled polypeptide binds to the RF present on the first particle and the labeled antibody binds to any anti-CCP antibodies on the second particle. The amount of label is subsequently detected and optionally quantified to determine the presence and/or amount of RF and anti-CCP antibodies. Because of this selection of capture and detection reagents, the reagents for capture and detection of anti-CCP antibodies do not interfere with the action of reagents for capture and detection of RF, and vice versa.

In some embodiments, the first particles and the second particles can be distinguished by flow cytometry by a characteristic independent of the presence or absence of the CCP and antibody immobilized on the first and second particles respectively. In these embodiments, the particles can be sorted and the amount of label associated with each particle can be determined, thereby allowing for simultaneous determination of the amount of anti-CCP antibodies and RF in the sample.

In some embodiments, the methods of the invention comprise a single reaction vessel comprising at least a first and second particle group, wherein the first particle group includes an immobilized CCP and the second particle group includes multiple immobilized capture antibodies that have specificities for two of human IgM, IgG, and IgA, or immobilized capture antibodies that have specificities for all three of human IgM, IgG, and IgA.

C. Detectable Labels

The labels used in the labeled binding members can be any label that is capable of directly or indirectly emitting or generating detectable signal. In some embodiments, the labels are fluorophores. As noted in more detail below, fluorophores may also be incorporated into the particles themselves to distinguish one group of particles from another. A vast array of fluorophores are reported in the literature and thus known to those skilled in the art, and many are readily available from commercial suppliers to the biotechnology industry. Literature sources for fluorophores include Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995).

The following is a list of examples of fluorophores:
4-acetamido-4′-isothiocyanatostilbene-2,2′disulfonic acid
acridine
acridine isothiocyanate
5-(2′-aminoethyl)aminonaphthalene-1-sulfonic acid (EDANS)
4-amino-N-[3-vinylsulfonyl)phenyl]naphthalimide-3,5 disulfonate
N-(4-anilino-1-naphthyl)maleimide
anthranilamide
BODIPY
Brilliant Yellow
coumarin
7-amino-4-methylcoumarin (AMC, Coumarin 120)
7-amino-4-trifluoromethylcoumarin (Coumaran 151)
cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo)benzoic acid (DABCYL)
4-dimethylaminophenylazophenyl-4′-isothiocyanate (DABITC)
eosin
eosin isothiocyanate
erythrosin B
erythrosin isothiocyanate
ethidium
5-carboxyfluorescein (FAM)
5-(4,6-dichlorotriazin-2-yl)aminofluorescein (DTAF)
2′,7′-dimethoxy-4′5′-dichloro-6-carboxyfluorescein (JOE)
fluorescein
fluorescein isothiocyanate
fluorescamnine
IR144
IR1446
Malachite Green isothiocyanate
4-methylumbelliferone
ortho cresolphthalein
nitrotyrosine
pararosaniline
Phenol Red
phycoerythrin (including but not limited to B and R types)
o-phthaldialdehyde
pyrene
pyrene butyrate
succinimidyl 1-pyrene butyrate
quantum dots
Reactive Red 4 (Cibacron™ Brilliant Red 3B-A)
6-carboxy-X-rhodamine (ROX)
6-carboxyrhodamine (R6G)
lissamine rhodamine B sulfonyl chloride rhodamine
rhodamine B
rhodamine 123
rhodamine X isothiocyanate
sulforhodamine B
sulforhodamine 101
sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)
riboflavin
rosolic acid
lanthanide chelate derivatives

The fluorophores (or other labels) can be used in combination, with a distinct label for each analyte. In some embodiments, however, a single label is used for all labeled binding members, the assays being differentiated solely by the differentiation parameter distinguishing the individual particle groups from each other.

The attachment of any of these fluorophores to the binding members described above to form assay reagents for use in the practice of this invention is achieved by conventional covalent bonding, using appropriate functional groups on the fluorophores and on the binding members. The recognition of such groups and the reactions to form the linkages will be readily apparent to those skilled in the art.

Methods of, and instrumentation for, flow cytometry are known in the art, and can be used in the practice of the present invention. Flow cytometry in general resides in the passage of a suspension of particles (or cells) in as a stream through a light beam and coupled to electro-optical sensors, in such a manner that only one particle at a time passes the region of the sensors. As each particle passes this region, the light beam is perturbed by the presence of the particle, and the resulting scattered and fluoresced light are detected. The optical signals are used by the instrumentation to identify the subgroup to which each particle belongs, along with the presence and amount of label, so that individual assay results are achieved. Descriptions of instrumentation and methods for flow cytometry are found in the literature. Examples are McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J. Immunol. Meth. 126: 183-189 (1990); McHugh, “Flow Cytometry and the Application of Microsphere-Based Fluorescence Immunoassays,” Immunochemica 5: 116 (1991); Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230 (1988); Fulwyler et al., “Flow Microsphere Immunoassay for the Quantitative and Simultaneous Detection of Multiple Soluble Analytes,” Meth. Cell Biol. 33: 613-629 (1990); Coulter Electronics Inc., United Kingdom Patent No. 1,561,042 (published Feb. 13, 1980); and Steinkamp et al., Review of Scientific Instruments 44(9): 1301-1310 (1973).

Similarly, methods of and instrumentation for applying and removing a magnetic field as part of an assay are known to those skilled in the art and reported in the literature. Examples of literature reports are Forrest et al., U.S. Pat. No. 4,141,687 (Technicon Instruments Corporation, Feb. 27, 1979); Ithakissios, U.S. Pat. No. 4,115,534 (Minnesota Mining and Manufacturing Company, Sep. 19, 1978); Vlieger, A. M., et al., Analytical Biochemistry 205:1-7 (1992); Dudley, Journal of Clinical Immunoassay 14:77-82 (1991); and Smart, Journal of Clinical Immunoassay 15:246-251 (1992). All of the citations in this and the preceding paragraph are incorporated herein by reference.

D. Solid Supports

Any type of solid support can be used in the invention. In some embodiments, the solid support is spherical or near-spherical. In some embodiments, the particles used in the practice of this invention are microscopic in size and formed of a polymeric material. Polymers that will be useful as microparticles are those that are chemically inert relative to the components of the biological sample and to the assay reagents other than the binding member coatings that are affixed to the microparticle surface. Suitable microparticle materials will also have minimal autofluorescence, will be solid and insoluble in the sample and in any buffers, solvents, carriers, diluents, or suspending agents used in the assay, and will be capable of affixing to the appropriate coating material. Examples of suitable polymers are polystyrenes, polyesters, polyethers, polyolefins, polyalkylene oxides, polyamides, polyurethanes, polysaccharides, celluloses, and polyisoprenes. Crosslinking is useful in many polymers for imparting structural integrity and rigidity to the microparticle. The size range of the microparticles can vary. In some embodiments, the microparticles range in diameter from about 0.3 micrometers to about 100 micrometers, e.g., from about 0.5 micrometers to about 40 micrometers, e.g., from about 2 micrometers to about 10 micrometers.

To facilitate the particle recovery and washing steps of the assay, the particles preferably contain a magnetically responsive material, i.e., any material that responds to a magnetic field. Separation of the solid and liquid phases, either after incubation or after a washing step, is then achieved by imposing a magnetic field on the reaction vessel in which the suspension is incubated, causing the particles to adhere to the wall of the vessel and thereby permitting the liquid to be removed by decantation or aspiration. Magnetically responsive materials of interest in this invention include paramagnetic materials, ferromagnetic materials, ferrimagnetic materials, and metamagnetic materials. Examples, include, e.g., iron, nickel, and cobalt, as well as metal oxides such as Fe₃O₄, BaFe₁₂O₁₉, CoO, NiO, Mn₂O₃, Cr₂O₃, and CoMnP.

The magnetically responsive material can be dispersed throughout the polymer, applied as a coating on the polymer surface or as one of two or more coatings on the surface, or incorporated or affixed in any other manner that secures the material in to the particle. The quantity of magnetically responsive material in the particle is not critical and can vary over a wide range. The quantity can affect the density of the microparticle, however, and both the quantity and the particle size can affect the ease of maintaining the microparticle in suspension for purposes of achieving maximal contact between the liquid and solid phase and for facilitating flow cytometry. An excessive quantity of magnetically responsive material in the microparticles may produce autofluorescence at a level high enough to interfere with the assay results. Therefore, in some embodiments, the concentration of magnetically responsive material is low enough to minimize any autofluorescence emanating from the material. With these considerations in mind, the magnetically responsive material in a particle in accordance with this invention is, for example, from about 0.05% to about 75% by weight of the particle as a whole. In some embodiments, the weight percent range is from about 1% to about 50%, e.g., from about 2% to about 25%, e.g., from about 2% to about 8%.

Coating of the particle surface with the appropriate assay reagent can be achieved by electrostatic attraction, specific affinity interaction, hydrophobic interaction, or covalent bonding. The polymer can be derivatized with functional groups for covalent attachment of the assay reagents by conventional means, notably by the use of monomers that contain the functional groups, such monomers serving either as the sole monomer or as a co-monomer. Examples of suitable functional groups are amine groups (—NH₂), ammonium groups (—NH₃⁺ or —NR₃⁺), hydroxyl groups (—OH), carboxylic acid groups (—COOH), and isocyanate groups (—NCO). Useful monomers for introducing carboxylic acid groups into polyolefins, for example, are acrylic acid and methacrylic acid.

Linking groups can be used as a means of increasing the density of reactive groups on the particle surface and decreasing steric hindrance. This may increase the range and sensitivity of the assay. Linking groups can also be used as a means of adding specific types of reactive groups to the solid phase surface if needed to secure the particular coating materials of this invention.

The CCP or antibody can be directly or indirectly linked to the solid support via a linking agent. The CCP or antibody and solid support can be conjugated via a single linking agent or multiple linking agents. For example, the CCP or antibody and solid support may be conjugated via a single multifunctional (e.g., bi-, tri-, or tetra-) linking agent or a pair of complementary linking agents. In some embodiments, the CCP or antibody and solid support are conjugated via two, three, or more linking agents. Suitable linking agents include, e.g., functional groups, affinity agents, stabilizing groups, and combinations thereof.

In some embodiments, an affinity agent (e.g., agents that specifically binds to a ligand) is the linking agent. In these embodiments, for example, a first linking agent is bound to the CCP or antibody and a second linking agent is bound to the solid support. Affinity agents include receptor-ligand pairs, antibody-antigen pairs and other binding partners such as streptavidin/avidin and biotin. In some embodiments, the first linking agent is biotin and the second linking agent is streptavidin or avidin. In some embodiments, the first linking agent is a hapten (e.g., fluorescein) and the second linking agent is an anti-hapten (e.g., anti-fluorescein) antibody.

Functional groups include monofunctional linkers comprising a reactive group as well as multifunctional crosslinkers comprising two or more reactive groups capable of forming a bond with two or more different functional targets (e.g., peptides, proteins, macromolecules, semiconductor nanocrystals, or substrate). In some embodiments, the multifunctional crosslinkers are heterobifunctional crosslinkers comprising two different reactive groups.

Suitable reactive groups include, e.g., thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulffhydryl (SH), phosphate (—PO₃), or photoreactive moieties. Amine reactive groups include, e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, e.g., haloacetyl and alkyl halide derivates, maleimides, aziridines, acryloyl derivatives, arylating agents, and thiol-disulfides exchange reagents. Carboxylate reactive groups include, e.g., diazoalkanes and diazoacetyl compounds, such as carbonyldiimidazoles and carbodiimides. Hydroxyl reactive groups include, e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, e.g., hydrazine derivatives for Schiff base formation or reduction amination. Active hydrogen reactive groups include, e.g., diazonium derivatives for Mannich condensation and iodination reactions. Photoreactive groups include, e.g., aryl azides and halogenated aryl azides, benzophenones, diazo compounds, and diazirine derivatives.

Other suitable reactive groups and classes of reactions useful in practicing the present invention are generally those that are well known in the art of bioconjugate chemistry. Currently favored classes of reactions available with reactive chelates are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

In some embodiments, the functional group is a heterobifunctional crosslinker comprising two different reactive groups that contain heterocyclic rings that can interact with peptides and proteins. For example, heterobifunctional crosslinkers such as N-[γ-maleimidobutyryloxy]succinimide ester (GMBS) or succinimidyl 4-[N-maleimidomethyl]cyclohexane-1-carboxylate (SMCC) comprise an amine reactive group and a thiol-reactive group that can interact with amino and thiol groups within peptides or proteins. Additional combinations of reactive groups suitable for heterobifunctional crosslinkers include, for example, carbonyl and sulfhydryl reactive groups; amine and photoreactive groups; sulfhydryl and photoreactive groups; carbonyl and photoreactive groups; carboxylate and photoreactive groups; and arginine and photoreactive groups. Examples of suitable useful linking groups are polylysine, polyaspartic acid, polyglutamic acid and polyarginine. N-hydroxysucciniinide (NHS), CMC 1-cyclohexyl-3-(2-morpholinoethyl)carbodiimide (CMC), N-Hydroxybenzotriazole (HOBt), and/or other crosslinking agents may be used.

In general, care should be taken to avoid the use of particles that exhibit high autofluorescence. Particles formed by conventional emulsion polymerization techniques from a wide variety of starting monomers are generally suitable since they exhibit at most a low level of autofluorescence. Conversely, particles that have been modified to increase their porosity and hence their surface area, i.e., those particles that are referred to in the literature as “macroporous” particles, are less desirable since they tend to exhibit high autofluorescence. A further consideration is that autofluorescence increases with increasing size and increasing percentage of divinylbenzene monomer.

Multiplexing with the use of microparticles in accordance with this invention can be achieved by designing each particle (i.e., the “first” particle, and the “second” particle, and if relevant, the “third” particle, and the “fourth” particle) to have a distinctive differentiation parameter, which renders that group distinguishable from the other groups by flow cytometry.

One example of a differentiation parameter is the particle diameter, the various particle groups being defined by nonoverlapping diameter subranges. The widths of the diameter subranges and the spacing between mean diameters of adjacent subranges are selected to permit differentiation of the subranges by flow cytometry, and will be readily apparent to those skilled in the use of and instrumentation for flow cytometry. In this specification, the term “mean diameter” refers to a number average diameter. In some embodiments, the subrange width is about ±5% CV or less of the mean diameter, where “CV” stands for “coefficient of variation” and is defined as the standard deviation of the particle diameter divided by the mean particle diameter, times 100 percent. The minimum spacing between mean diameters among the various subranges can vary depending on the microparticle size distribution, the ease of segregating microparticles by size for purposes of attaching different assay reagents, and the type and sensitivity of the flow cytometry equipment. In some embodiments, best results will be achieved when the mean diameters of different subranges are spaced apart by at least about 6% of the mean diameter of one of the subranges, e.g., at least about 8% of the mean diameter of one of the subranges, e.g., at least about 10% of the mean diameter of one of the subranges. In some embodiments, the standard deviation of the particle diameters within each subrange is less than one third of the separation of the mean diameters of adjacent subranges.

Another example of a differentiation parameter that can be used to distinguish among the various groups of particles is fluorescence. Differentiation is accomplished by incorporating one or more fluorescent materials in the particles, the fluorescent materials having different fluorescent emission spectra and being distinguishable on this basis.

Fluorescence can in fact be used both as a means of distinguishing the particle groups from each other and as a means of detection and quantification for the assay performed on the particles. The use of fluorescent materials with different emission spectra can serve as a means of distinguishing the particle groups from each other and also as a means of distinguishing the particle group's classification from the (e.g., fluorescent) assay reported signals. An example of a fluorescent substance that can be used as a means of distinguishing particle groups is fluorescein and an example of a substance that can be used for the assay detection is phycoerythrin. In the use of this example, different particle groups can be dyed with differing concentrations of fluorescein to distinguish them from each other, while phycoerythrin is used as the label on the various labeled binding members used in the assay.

Still other examples of a differentiation parameter that can be used to distinguish among the various groups of particles are light scatter, or a combination of light scatter. Side angle light scatter varies with particle size, granularity, absorbance and surface roughness, while forward angle light scatter is mainly affected by size and refractive index. Thus, varying any of these qualities can serve as a means of distinguishing the various groups. Light emission can be varied by incorporating fluorescent materials in the microparticles and using fluorescent materials that have different fluorescence intensities or that emit fluorescence at different wavelengths, or by varying the amount of fluorescent material incorporated. By using fluorescence emissions at different wavelengths, the wavelength difference can be used to distinguish the particle groups from each other, while also distinguishing the labels in the labeled binding members from the labels that differentiate one particle group from another.

In a variation of the above, the microparticles will have two or more fluorochromes incorporated within them so that each microparticle in the array will have at least three differentiation parameters associated with it, i.e., light scatter together with fluorescent emissions at two separate wavelengths. For example, the microparticle can be made to contain a red fluorochrome such as Cy5 together with a far-red fluorochrome such as Cy5.5. Additional fluorochromes can be used to further expand the system. Each microparticle can thus contain a plurality of fluorescent dyes at varying wavelengths.

Still another example of a differentiation parameter that can be used to distinguish among the various groups of particles is absorbance. When light is applied to microparticles the absorbance of the light by the particles is indicated mostly by the strength of the laterally (side-angle) scattered light while the strength of the forward-scattered light is relatively unaffected. Consequently, the difference in absorbance between various colored dyes associated with the raicroparticles is determined by observing differences in the strength of the laterally scattered light.

A still further example of a differentiation parameter that can be used to distinguish among the various groups of particles is the number of particles in each group. The number of particles of each group is varied in a known way, and the count of particles having various assay responses is determined. The various responses are associated with a particular assay by the number of particles having each response.

As the above examples illustrate, a wide array of parameters or characteristics can be used as differentiation parameters to distinguish the microparticles of one group from those of another. The differentiation parameters may arise from particle size, from particle composition, from particle physical characteristics that affect light scattering, from excitable fluorescent dyes or colored dyes that impart different emission spectra and/or scattering characteristics to the microparticles, or from different concentrations of one or more fluorescent dyes. When the distinguishable microparticle parameter is a fluorescent dye or color, it can be coated on the surface of the microparticle, embedded in the microparticle, or bound to the molecules of the microparticle material. Thus, fluorescent microparticles can be manufactured by combining the polymer material with the fluorescent dye, or by impregnating the microparticle with the dye. Microparticles with dyes already incorporated and thereby suitable for use in the present invention are commercially available, from suppliers such as Spherotech, Inc. (Libertyville, Ill., USA) and Molecular Probes, Inc. (Eugene, Oreg., USA).

D. Diagnosis

As described herein, the methods of the present invention allow for the simultaneous detection and optionally quantification of anti-CCP and RF in a sample. The presence of CCP and RF are associated with rheumatoid arthritis and in the case of RF, other autoimmune diseases as well. Therefore, in one aspect of the invention, a sample is obtained from an individual, the quantity of RF and anti-CCP antibodies are determined as described herein, and then the amount of RF and anti-CCP antibodies are used to determine a prognosis or diagnosis of an autoimmune disease, including but not limited to, rheumatoid arthritis, and for RF, also, e.g., systemic lupus erythematosus (SLE), Sjogren's syndrome, scleroderma, and polymyositis. In some embodiments, the detection and/or quantification of RF and anti-CCP antibodies in a sample is used to provide a prognosis or to assess the efficacy of a pharmaceutical (e.g., anti-arthritis drug) treatment. Diagnosis, prognosis, or assessing pharmaceutical efficacy can be achieved for example by correlating the amount of RF and/or anti-CCP antibodies in a sample with known amounts associated with, e.g., healthy and/or diseased individuals.

### III. Reaction Mixtures

The present invention also provides for reaction mixtures. Such mixtures comprise one or more of the components of the above-described method in the same aqueous reaction mixture. Thus, for example, the reaction mixture optionally comprises a biological sample in addition to a cyclic citrullinated peptide (CCP) immobilized on a first solid support (e.g., a first particle) and an antibody immobilized on a second solid support (e.g., a second particle), wherein the antibody has a binding specificity for human IgM, IgG or IgA. In some embodiments, the antibody immobilized on the second particle is an antibody with binding specificity for human IgM. In some embodiments, the antibody immobilized on the second particle is an antibody with binding specificity for human IgG. In some embodiments, the antibody immobilized on the second particle is an antibody with binding specificity for human IgA. In some embodiments, the kits comprise a second and third and optionally a fourth particle, where each of the second and third and fourth particles have immobilized a different antibody binding specificity for human IgM, IgG or IgA.

The reaction mixture can further comprise a labeled polypeptide comprising an Fc region of a non-human antibody, as described herein, and a labeled antibody that binds to human IgG, also as described herein. In some embodiments, the particles are magnetically responsive particles.

In some embodiments, the first and second particles comprise different dyes such that the first and second particles are distinguishable by flow cytometry.

In some embodiments, the Fc region of the labeled polypeptide is a non-human Fc region. For example, in some embodiments, the Fc region is a horse or goat Fc region.

In some embodiments, the labeled antibody is a non-human antibody. For example, in some embodiments, the labeled antibody is a donkey or mouse antibody.

### IV. Kits

The present invention also provides for kits of performing the methods of the invention as described herein and can include any combination of the reagents described herein. For example, in some embodiments, the kit comprises a cyclic citrullinated peptide (CCP) immobilized on a first solid support (e.g., a first particle) and an antibody immobilized on a second solid support (e.g., a second particle), wherein the antibody has a binding specificity for human IgM, IgG or IgA. In some embodiments, the antibody immobilized on the second particle is an antibody with binding specificity for human IgM. In some embodiments, the antibody immobilized on the second particle is an antibody with binding specificity for human IgG. In some embodiments, the antibody immobilized on the second particle is an antibody with binding specificity for human IgA. In some embodiments, the kits comprise a second and third and optionally a fourth particle, where each of the second and third and fourth particles have immobilized a different antibody binding specificity for human IgM, IgG and/or IgA.

The kit can further comprise a labeled polypeptide comprising an Fc region of a non-human antibody, as described herein, and a labeled antibody that binds to human IgG, also as described herein. In some embodiments, the particles are magnetically responsive particles.

In some embodiments, the first and second particles comprise different dyes such that the first and second particles are distinguishable by flow cytometry.

In some embodiments, the Fc region of the labeled polypeptide is a non-human Fc region. For example, in some embodiments, the Fc region is a horse or goat Fc region.

In some embodiments, the labeled antibody is a non-human antibody. For example, in some embodiments, the labeled antibody is a donkey or mouse antibody.

The kits of the invention can optionally include assay controls, e.g., a known amount of RF and/or anti-CCP antibodies. Such controls are useful as positive or negative for example.

The kit can also include instructions for use, which can be in paper or electronic (e.g., disk or other computer readable) form.

In some embodiments, the multiplexed assays of anti-CCP and RF may be automated with the appropriate use of instrumentation.

### Examples

The following examples are offered to illustrate, but not to limit the claimed invention.

### Example 1

The particles and other materials used in the example were prepared as follows:

The buffer solutions used in these examples are as follows:

| | |
|---|---|
| Wash | Phosphate buffer Saline pH 7.4 plus tween 20 and sodium |
| Buffer | azide (<0.1%) as preservatives. |
| Particle | Glycerol and protein stabilizers (bovine) in a MOPS |
| Diluent | (3-[N-Morpholino] propanesulfonic acid) buffer |
| | Proclin ® 300 (0.3%) and sodium azide |
| | (<0.1%) as preservatives |
| Sample | Protein stabilizers (bovine and murine) in a triethanolamine |
| Diluent | buffer Proclin ® 300 (0.3%) and sodium azide |
| | (<0.1%) as preservatives |
| Conjugate | Protein stabilizer (bovine) in a phosphate buffer |
| Diluent | Proclin ® 300 (0.3%) and sodium azide (<0.1%) |
| | as preservatives |

Coating of Particles with CCP (Cyclic Citrullinated Peptide)

Bead stock at a concentration of approximately 10 mg/ml was placed into a polypropylene centrifuge tube. The beads were washed several times with an Ethanol/MES (2-(N-morpholino)-ethane-sulfonic acid), pH 6.1 solution. After mixing, the mixture was placed on a magnet until the supernatant was clear and the solution was aspirated.

NHS (N-hydroxysuccinimide) and CMC (1-cyclohexyl-3-(2-morpholinoethyl) carbodilmide) solutions, which were prepared using an Ethanol/MES, pH 6.1 buffer, were added to the beads. The bead mixture was immediately vortex and allowed to mix on a roller for 30 minutes. The bead mixture was placed on a magnet until the supernatant was clear. The supernatant was aspirated and the beads were washed several times by adding an Ethanol/Sodium phosphate, pH 5.0 solution.

Beads were resuspended in a Sodium Phosphate, pH 5.0 solution. Streptavidin in a phosphate buffered saline (PBS), pH 7.4 solution was added to the bead suspension. The bead mixture was immediately vortexed and allowed to mix on a roller for 60 minutes. The beads were separated on a magnet and the supernatant was removed. The Streptavidin coated beads were washed with PBS, pH 7.4 solution. After the final wash the solution was aspirated, the beads were resuspended in a PBS, pH 7.4 solution. A solution of CCP-Biotin in PBS, pH 7.4 was then added to the resuspended beads. This solution was allowed to mix for 15 minutes. The beads were placed on a magnet and the solution was aspirated. Beads were washed several times with a Tween/PBS, pH 7.4 solution. Upon final aspiration, a blocking solution was added to the beads and allowed to mix for 2 hours. The beads were separated and washed several times with particle diluent and stored in particle diluent for future multiplexing.

Coating of Particles with Donkey Anti-Human IgM (DAHM)

Bead stock at a concentration of approximately 10 mg/ml was placed into a polypropylene centrifuge tube. The beads were washed several times with an Ethanol/MES (2-(N-morpholino)-ethane-sulfonic acid), pH 6.1 solution. After mixing, the mixture was placed on a magnet until the supernatant was clear and the solution was aspirated.

NHS (N-hydroxysuccinimide) and CMC (1-cyclohexyl-3-(2-morpholinoethyl) carbodiimide) solutions, which were prepared using an Ethanol/MES, pH 6.1 buffer, were added to the beads. The bead mixture was immediately vortexed and allowed to mix on a roller for 30 minutes. The bead mixture was placed on a magnet until the supernatant was clear. The supernatant was aspirated and the beads were washed several times by adding an Ethanol/Sodium phosphate, pH 5.0 solution.

Beads were resuspended in a Sodium Phosphate, pH 5.0 solution. A solution of Donkey Anti-Human IgM in PBS, pH 7.4 was then added to the resuspended beads. This solution was allowed to mix for 60 minutes. The beads were placed on a magnet and the solution was aspirated. Beads were washed several times with a Tween/PBS, pH 7.4 solution. Upon final aspiration, a blocking solution was added to the beads and allowed to mix for 2 hours. The beads were separated and washed several times with particle diluent and stored in particle diluent for future multiplexing.

### Conjugation of Mouse Anti-Human IgG-PE and Equine-FC-PE

To conjugate B-Phycoerythrin (B-PE) to antibodies, many different cross-linkers and conjugation approaches can be used (Bioconjugate Techniques, Hermanson, G. T., Academic Press, 1996). For both the Fc fragment of equine IgG (eIgGFc) and monoclonal antibody to human IgG, we chose to use a combination of (1) activation of B-PE with SMCC (succinimidyl 4-[N-maleimidomethyl]-cyclohexane-1-carboxylate) cross-linker and (2) activation of antibody with SATA (N-succinimidyl S-acetylthioacetate)/hydroxylamine cross-linker.

B-PE was conjugated with SMCC to introduce thiol(—SH) reactive, maleimido moieties in B-PE (with input molar ratio of approximately 1:25=B-PE:SMCC). Then, the antibody, either mAb to human IgG or eIgGFc, was conjugated with SATA (with input molar ratio of approximately 1:15=Ab:SATA), followed by removal of S-acetyl group in SATA [with addition of excess hydroxylamine (NH2OH)] to produce free —SH groups in the antibody (internal disulfide groups of the antibody are unaffected). The SMCC activated B-PE was then mixed with the reactive antibody-SATA for the conjugation reaction at 3/1 antibody/B-PE molar ratio. The thiol group of SATA in the antibody reacts with the maleimido moiety of SMCC in B-PE to produce a stable thio-ether linkage between B-PE and the antibody, resulting in stable B-PE/antibody conjugate. The final B-PE/antibody conjugate was purified by Sephacryl S-300HR size exclusion chromatography.

### Multiplex Assay Procedure for CCP and RF

The CCP and Donkey anti-human IgM beads along with three QC beads are diluted in particle diluent to a concentration of approximately 10 μg/ml. The multiplexed bead reagent is placed into a reagent pack for use on the BioPlex 2200 along with sample diluent and a conjugate reagent that contains mouse anti-human IgG-PE, horse IgG Fc-PE and anti-FXIII PE, which is used for the SVB QC assay. The BioPlex 2200 System combines a 5 μL aliquot of patient sample, 100 μL sample diluent, 195 μL wash buffer from the instrument and 100 μL bead reagent into a reaction vessel. The mixture is incubated at 37° C. After a wash cycle, 50 μL of conjugate reagent is added to the dyed beads and this mixture is incubated at 37° C. The anti-human IgG PE binds to anti-CCP IgG antibodies that are bound to the CCP coated bead and the horse IgG Fc antibodies bind to the IgM Rheumatoid Factor antibodies that are captured by the anti-human IgM coated beads. The excess conjugate is removed in another wash cycle, and the beads are re-suspended in wash buffer. The bead mixture then passes through the detector. The identity of the dyed beads in determined by the fluorescence of the dyes, and the amount of antibody captured by the antigen is determined by the fluorescence of the attached PE. Raw data is calculated in relative fluorescence intensity (RFI).

Three additional dyed beads, an Internal Standard Bead (ISB), a Serum Verification Bead (SVB) and a Reagent Blank Bead (RBB) are present in each reaction mixture to verify detector response, the addition of serum to the reaction vessel and the absence of significant non-specific binding in serum.

The instrument is calibrated using a set of six distinct calibrator vials. The result of each of these antibodies is expressed as IU/ml.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for analyzing the level of rheumatoid factor (RF) and anti-cyclic citrullinated peptide (CCP) antibodies in a sample from a human, the method comprising
a) incubating the sample with a mixture of particles in a first suspension, said mixture of particles comprising:
(i) a cyclic citrullinated peptide (CCP) immobilized on a first particle; and
(ii) an antibody immobilized on a second particle, wherein the antibody has a binding specificity for human IgM, IgG or IgA,
wherein the incubation of step a) is performed under conditions to allow anti-CCP antibodies and RF antibodies, if present in the sample, to bind to the CCP and the antibody, respectively;
b) recovering said particles from said first suspension; and
c) incubating said recovered particles with a mixture of labeled binding members, the members comprising:
(i) a labeled polypeptide comprising an Fc region of an antibody; and
(ii) a labeled antibody that binds human IgG,
wherein the incubation of step c) is performed under conditions to allow the labeled polypeptide and the labeled antibody to bind the bound RF antibodies and the bound anti-CCP antibodies, respectively, if present; and
d) recovering said particles from said second suspension; and
e) detecting the amount of label bound to said particles thus recovered from said second suspension, thereby obtaining values individually representative of the levels of rheumatoid factor and anti-cyclic citrullinated peptide.

2. The method of claim 1, wherein the particles in section a) parts (i) and (ii) are distinguishable by a flow cytometry distinguishable characteristic that is independent of the CCP or antibody immobilized on the particles; and
step e) comprises detecting the amount of label bound to said particles recovered from said second suspension while correlating by flow cytometry the amount of label thus detected to the particle to which label is bound.

3. The method of claim 1, wherein the sample is a blood, plasma, or serum sample.

4. The method of claim 1, wherein step a) comprises incubating an antibody having binding specificity for human IgM immobilized on the second particle.

5. The method of claim 1, wherein step a) comprises incubating an antibody having binding specificity for a human IgG immobilized on the second particle.

6. The method of claim 1, wherein step a) comprises incubating an antibody having binding specificity for human IgA immobilized on the second particle.

7. The method of claim 1, wherein step a) comprises incubating an antibody having binding specificity for human IgG immobilized on the second particle and an antibody having binding specificity for a human IgM immobilized on the second particle.

8. The method of claim 1, wherein step a) comprises incubating an antibody having binding specificity for human IgA immobilized on the second particle and an antibody having binding specificity for a human IgM immobilized on the second particle.

9. The method of claim 1, wherein step a) comprises incubating an antibody having binding specificity for human IgA immobilized on the second particle and an antibody having binding specificity for a human IgG immobilized on the second particle.

10. The method of claim 1, wherein step a) comprises incubating an antibody having binding specificity for human IgA immobilized on the second particle, an antibody having binding specificity for a human IgM immobilized on the second particle, and an antibody having binding specificity for human IgG immobilized on the second particle.

11. The method of claim 1, wherein step a) comprises incubating a first antibody having binding specificity for one of the group consisting of human IgA, IgG and IgM immobilized on the second particle and a second antibody having binding specificity for one of the group consisting of human IgA, IgG and IgM immobilized on a third particle, wherein the second antibody binds to a different member of the group than the first antibody.

12. The method of claim 1, wherein step a) comprises incubating a first antibody having binding specificity for one of the group consisting of human IgA, IgG and IgM immobilized on the second particle, a second antibody having binding specificity for one of the group consisting of human IgA, IgG and IgM immobilized on a third particle, and a second antibody having binding specificity for one of the group consisting of human IgA, IgG and IgM immobilized on a third particle, wherein each of the first, second and third antibodies bind to a different member of the group.

13. The method of claim 1, wherein the particles are magnetically-responsive particles and steps b) and d) comprise subjecting the first and second suspensions, respectively, to a magnetic field to cause the particles to adhere to a reaction vessel wall.

14. The method of claim 1, wherein the labels are fluorescent labels.

15. The method of claim 1, wherein the first and second particles comprise different dyes such that the first and second particles are distinguishable by flow cytometry, and wherein step e) comprises distinguishing the first and second particles by flow cytometry.

16. The method of claim 1, wherein said Fc region is a non-human Fc region and/or said antibody immobilized on said second particle is a non-human antibody and/or said labeled antibody that binds human IgG is a non-human antibody.

17. The method of claim 1, wherein said Fc region is a horse Fc region and/or said antibody immobilized on said second particle is a donkey antibody and/or said labeled antibody that binds human IgG is a mouse antibody.

18. The method of claim 1, wherein the particles in the mixture are approximately the same diameter.

19. The method of claim 1, wherein the first and second particles have sufficiently different diameter to be distinguishable by flow cytometry.

20. A kit for detecting factors associated with rheumatoid arthritis, the kit comprising,
a) a cyclic citrullinated peptide (CCP) immobilized on a first solid support; and
b) an antibody immobilized on a second solid support, wherein the antibody has a binding specificity for human IgM, IgG or IgA.

21. The kit of claim 20, wherein the solid supports of a) and b) are in the same reaction vessel.

22. The kit of claim 20, wherein the solid supports in a) and b) are particles.

23. The kit of claim 20, wherein the solid supports in a) and b) are different particles.

24. The kit of claim 22 or 23, wherein the particles are magnetically-responsive particles.

25. The kit of claim 20, wherein the antibody immobilized on the second solid support is a non-human antibody.

26. The kit of claim 20, further comprising,
c) a labeled antibody that binds human IgG; and/or
d) a labeled polypeptide comprising an Fc region of an antibody.

27. The kit of claim 26, wherein the labels are fluorescent labels.

28. The kit of claim 22, wherein the first and second solid supports comprise different dyes such that the first and second solid supports are distinguishable by flow cytometry.

29. The kit of claim 26, wherein said Fc region is a non-human Fc region and/or said labeled antibody that binds human IgG is a non-human antibody.

30. The kit of claim 26, wherein said Fc region is a horse Fc region and/or said antibody immobilized on said second particle is a donkey antibody and/or said labeled antibody that binds human IgG is a mouse antibody.

31. The kit of claim 20, further comprising an assay control comprising an amount of anti-CCP antibodies and/or an amount of RF.

32. A reaction mixture comprising,
a) a cyclic citrullinated peptide (CCP) immobilized on a first solid support; and
b) an antibody immobilized on a second solid support, wherein the antibody has a binding specificity for human IgM, IgG or IgA.

33. The reaction mixture of claim 32, further comprising a biological sample.

34. The reaction mixture of claim 32, wherein the solid supports in a) and b) are particles.

35. The reaction mixture of claim 32, wherein the solid supports in a) and b) are different particles.

36. The reaction mixture of claim 34 or 35, wherein the particles are magnetically-responsive particles.

37. The reaction mixture of claim 32, wherein the antibody immobilized on the second solid support is a non-human antibody.

38. The reaction mixture of claim 32, further comprising,
c) a labeled antibody that binds human IgG; and/or
d) a labeled polypeptide comprising an Fc region of an antibody.

39. The reaction mixture of claim 38, wherein the labels are fluorescent labels.

40. The reaction mixture of claim 34, wherein the first and second solid supports comprise different dyes such that the first and second particles are distinguishable by flow cytometry.

41. The reaction mixture of claim 38, wherein said Fc region is a non-human Fc region and/or said labeled antibody that binds human IgG is a non-human antibody.

42. The reaction mixture of claim 38, wherein said Fc region is a horse Fc region and/or said antibody immobilized on said second particle is a donkey antibody and/or said labeled antibody that binds human IgG is a mouse antibody.

43. A method for analyzing the level of rheumatoid factor (RF) and anti-cyclic citrullinated peptide (CCP) antibodies in a sample from a human, the method comprising
a) incubating the sample with a mixture of particles in a first suspension, said mixture of particles comprising:
(i) a first capture agent immobilized on a first particle, said first capture agent capable of binding a human anti-CCP antibody; and
(ii) a second capture agent immobilized on a second particle, said second capture agent capable of binding a human anti-RF antibody,
wherein the incubation of step a) is performed under conditions to allow anti-CCP antibodies and RF antibodies, if present, to bind to the first capture agent and the second capture agent, respectively;
b) recovering said particles from said first suspension; and
c) incubating said recovered particles with a mixture of labeled binding members, the members comprising:
(i) a labeled first detection agent that binds anti-CCP antibodies; and
(ii) a labeled second detection agent that binds anti-RF antibodies,
wherein the incubation of step c) is performed under conditions to allow the labeled first detection agent and the labeled second detection agent to bind the bound anti-CCP antibodies and the bound RF antibodies and, respectively, if present; and
d) recovering said particles from said second suspension; and
e) detecting the amount of label bound to said particles thus recovered from said second suspension, thereby obtaining values individually representative of the levels of rheumatoid factor and anti-cyclic citrullinated peptide.

44. The method of claim 43, wherein:
the first capture agent comprises a CCP; and
the first detection agent comprises an antibody that binds to human IgG.

45. The method of claim 43, wherein
the first capture agent comprises an antibody that binds to human IgG; and
the first detection agent comprises a CCP.

46. The method of claim 43, wherein
the second capture agent comprises an Fc region of an antibody;
and the second detection agent comprises an antibody having binding specificity for a human IgM, IgG, or IgA.

47. The method of claim 43, wherein
the second capture agent comprises an antibody having binding specificity for a human IgM, IgG, or IgA; and
the second detection agent comprises an Fc region of an antibody.

48. The method of claim 44, wherein the incubating step a) comprises incubating an exogenous anti-CCP antibody that competes with an anti-CCP antibody in the sample, and the labeled first detection agent binds to the exogenous anti-CCP antibody but does not bind to the anti-CCP antibody in the sample.

49. The method of claim 46, wherein the incubating step a) comprises incubating an exogenous anti-FC antibody that competes with an anti-RF antibody in the sample, and the labeled first detection agent binds to the exogenous anti-FC antibody but does not bind to the anti-RF antibody in the sample.
